(19) United States
(12) Patent Application Publication
(10) Pub. No.: US 2026/0185991 A1
(43) Pub. Date: Jul. 2, 2026

(54) Devices for Detection and Differentiation of Renal Disease

(71) Applicant: IDEXX Laboratories, Inc., Westbrook, ME (US)

(72) Inventor: Murthy Yerramilli, Westbrook, ME (US)

(21) Appl. No.: 19/423,906

(22) Filed: Dec. 17, 2025

Related U.S. Application Data
(60) Provisional application No. 63/740,725, filed on Dec. 31, 2024.

(51) Int. Cl. G01N 33/543; G01N 33/573; G01N 33/68

(52) U.S. Cl. CPC G01N 33/54388; G01N 33/573; G01N 33/6863; G01N 33/6893

(57) ABSTRACT

Disclosed are devices that comprise a solid phase, first and second analyte sensing reagents that are not specific for the same analyte, and that are incorporated in various architectures, and that find use in the detection of renal disease in a subject.

### RELATED APPLICATIONS

This application claims priority to U.S. Provisional Application No. 63/740,725 filed Dec. 31, 2024 which is incorporated herein by reference in its entirety.

### BACKGROUND

Renal disease, or chronic kidney disease (CKD) is a condition characterized by a gradual loss of kidney function over time. CKD can be present in a stable form or a progressive form each of which can be glomerular or tubular. As CKD worsens, metabolic waste products can build to high levels in the blood and lead to conditions such as high blood pressure, anemia, weak bones, poor nutritional health and nerve damage. CKD also increases the risk of heart and blood vessel disease and can eventually lead to kidney failure. CKD can be caused by a number of diseases and conditions and is commonly associated with diabetes and high blood pressure. Early detection and treatment can often slow progression of the disease and keep associated clinical conditions from worsening.

In humans, CKD progression is divided into five stages. In companion animals, such as dogs and cats, the stages of CKD are well known and have been established by the International Renal Interest Society (“IRIS”), and include Stages I (non-azotemic CKD), II (mild renal azotemia), III (moderate renal azotemia), and IV (severe renal azotemia). These stages are typically marked by serum concentrations of creatinine and SDMA, with changes in these functional kidney markers over time indicative of CKD. Nevertheless, using these markers to determine whether a subject has stable or progressive CKD is problematic primarily because these markers need to be monitored over a time course that creates the potential for irreversible kidney damage to occur during the evaluation period. Further, blood creatinine level is relatively insensitive as an early indicator of CKD, and can vary based on the subject's muscle mass and fasted state. SDMA can also present challenges as an accurate marker for CKD because it can be elevated in animals having diseases such as hyperthyroidism, and baseline SDMA levels can have significant differences between different breeds of animals (dogs and cats). As such, there remains a need for a diagnostic marker or a set of two or more diagnostic markers that provides accurate assessment of kidney function and injury status based on a single measurement would greatly improve the prevention, delay of onset, delay of progression, and or treatment of kidney disease including, for example, stable CKD, progressive CKD, and AKI.

While a number of devices, assays, and methods exist that provide for the quantitative and/or qualitative determination of one or more target analyte(s) in a biological sample. Many systems and methods comprise complicated or time consuming steps, cannot be transported (i.e., are not available as point-of-care systems), require substantial sample handling and/or preparation, and are not readily adaptable to multiplex assays and methods. The multiplexed devices, systems, assays, and methods described provide for greater flexibility in specifically targeting multiple selected analytes, with the availability of point-of-care analysis, and provide for the rapid identification, prognosis, diagnosis, and treatment of renal disease.

### SUMMARY

In one aspect, the disclosure provides a device comprising:
a solid phase having bound thereto at least a first analyte sensing reagent and a second analyte sensing reagent:
   A) the first analyte sensing reagent comprising a sensing reagent or an antibody specific for a first biomarker comprising:
      (i) Neutrophil Gelatinase-Associated Lipocalin (NGAL), an NGAL analog, or an antibody specific for NGAL, or any combination thereof;
      (ii) Symmetrical Dimethylarginine (SDMA), an SDMA analog, or an antibody specific for SDMA, or any combination thereof; or
      (ii) Cystatin B (CysB), a CysB analog, or an antibody specific for CysB, or any combination thereof;
   B) the second analyte sensing reagent comprising a sensing reagent or an antibody specific for a second biomarker; and
wherein the first analyte sensing reagent and the second analyte reagent are not the same.

In another aspect, the disclosure provides a device comprising:
a solid phase having bound thereto at least a first analyte sensing reagent and a second analyte sensing reagent:
   A) the first analyte sensing reagent comprising a sensing reagent or an antibody specific for a first biomarker comprising:
      (i) Neutrophil Gelatinase-Associated Lipocalin (NGAL), an NGAL analog, or an antibody specific for NGAL, or any combination thereof;
      (ii) Symmetrical Dimethylarginine (SDMA), an SDMA analog, or an antibody specific for SDMA, or any combination thereof; or
      (ii) Cystatin B (CysB), a CysB analog, or an antibody specific for CysB, or any combination thereof;
   B) the second analyte sensing reagent comprising a sensing reagent or an antibody specific for a second biomarker;
a first flow path and a second flow path, wherein the first flow path allows for a biological sample entry and delivery to the solid phase, which is independent from the second flow path; the second fluid path comprises a material essentially impermeable to said biological sample and which, upon contact with a wash reagent, becomes permeable to said biological sample and defines the second flow path for said biological sample;
a wash reagent that is permeable to said second fluid path material, which upon contact with said second fluid path material flows through said second fluid path material;
wherein the solid phase comprises a zone permeable to said biological sample, at the intersection between said first and second flow paths, whereby said biological sample initially flows only along said first flow path and said solid phase zone into said solid phase but not into said second fluid path material until said second fluid path material is rendered permeable by said wash reagent; and
wherein the first analyte sensing reagent and the second analyte reagent are not the same.

In another aspect, the disclosure provides a device comprising:
a solid phase having bound thereto at least a first analyte sensing reagent and a second analyte sensing reagent:
   A) the first analyte sensing reagent comprising a sensing reagent or an antibody specific for a first biomarker comprising:
      (i) Neutrophil Gelatinase-Associated Lipocalin (NGAL), an NGAL analog, or an antibody specific for NGAL, or any combination thereof;
      (ii) Symmetrical Dimethylarginine (SDMA), an SDMA analog, or an antibody specific for SDMA, or any combination thereof; or
      (ii) Cystatin B (CysB), a CysB analog, or an antibody specific for CysB, or any combination thereof;
   B) the second analyte sensing reagent comprising a sensing reagent or an antibody specific for a second biomarker;
a surface that comprises a single channel in fluid communication with a sample port, wherein the surface comprises a circular flow path at the peripheral edge of the surface, wherein the sample port and/or the channel comprises the solid phase; and
wherein the first analyte sensing reagent and the second analyte sensing reagent are not the same.

In some embodiments of the above aspect, the sample port and/or the channel comprises a conjugate comprising the first and/or the second analyte sensing reagent, and wherein the circular flow path comprises an immobilized capture ligand capable of binding at least one of the first and/or second analyte-analyte sensing reagent complex(es).

In another aspect, the disclosure provides a device comprising:
a solid phase having bound thereto at least a first analyte sensing reagent and a second analyte sensing reagent:
   A) the first analyte sensing reagent comprising a sensing reagent or an antibody specific for a first biomarker comprising:
      (i) Neutrophil Gelatinase-Associated Lipocalin (NGAL), an NGAL analog, or an antibody specific for NGAL, or any combination thereof;
      (ii) Symmetrical Dimethylarginine (SDMA), an SDMA analog, or an antibody specific for SDMA, or any combination thereof; or
      (ii) Cystatin B (CysB), a CysB analog, or an antibody specific for CysB, or any combination thereof;
   B) the second analyte sensing reagent comprising a sensing reagent or an antibody specific for a second biomarker;
a microfluidic cartridge comprising a base part, having a first face and a second opposite face and with a recess in the first face and a foil fixed to the base part to cover the recess and to form a foil face of said microfluidic cartridge, wherein the base part with the recess and the foil forms a flow channel and a sink, the flow channel has a length and comprises a reaction section and an upstream end and a downstream end, wherein the sink is in fluid communication with the flow channel downstream to the reaction section and the microfluidic cartridge comprises an inlet opening into said flow channel upstream to the reaction section and wherein said reaction section comprises the solid phase; and
wherein the first analyte sensing reagent and the second analyte sensing reagent are not the same.

In another aspect, the disclosure provides a device comprising:
a solid phase having bound thereto at least a first analyte sensing reagent and a second analyte sensing reagent:
   A) the first analyte sensing reagent comprising a sensing reagent or an antibody specific for a first biomarker comprising:
      (i) Neutrophil Gelatinase-Associated Lipocalin (NGAL), an NGAL analog, or an antibody specific for NGAL, or any combination thereof;
      (ii) Symmetrical Dimethylarginine (SDMA), an SDMA analog, or an antibody specific for SDMA, or any combination thereof; or
      (ii) Cystatin B (CysB), a CysB analog, or an antibody specific for CysB, or any combination thereof;
   B) the second analyte sensing reagent comprising a sensing reagent or an antibody specific for a second biomarker;
a planar waveguide with a planar surface comprising the solid phase; a refractive volume for optically coupling a light beam to the planar waveguide, the refractive volume refracting the light beam such that all of the light beam focuses at a non-zero, internal propagation angle at the planar surface;
a sample chamber for containing the sample in contact with the solid phase; and
wherein the first analyte sensing reagent and the second analyte sensing reagent are not the same.

In another aspect, the disclosure provides a device that comprises a cartridge comprising:
a solid phase having bound thereto at least a first analyte sensing reagent and a second analyte sensing reagent:
   A) the first analyte sensing reagent comprising a sensing reagent or an antibody specific for a first biomarker comprising:
      (i) Neutrophil Gelatinase-Associated Lipocalin (NGAL), an NGAL analog, or an antibody specific for NGAL, or any combination thereof;
      (ii) Symmetrical Dimethylarginine (SDMA), an SDMA analog, or an antibody specific for SDMA, or any combination thereof; or
      (ii) Cystatin B (CysB), a CysB analog, or an antibody specific for CysB, or any combination thereof;
   B) the second analyte sensing reagent comprising a sensing reagent or an antibody specific for a second biomarker;
a plurality of electrodes;
at least one magnet;
a first surface of the solid phase that transports a droplet, via the plurality of electrodes, on the first surface of the solid phase of the cartridge, wherein the droplet comprises at least one paramagnetic, bar-coded bead, and wherein the plurality of electrodes is configured to transport the droplet on the first surface of the solid phase the cartridge; and
a second surface of the solid phase that immobilizes the droplet, via the at least one magnet, on the second surface, wherein the at least one magnet is configured to immobilize the droplet on the second surface of the cartridge; and
wherein the first analyte sensing reagent and the second analyte sensing reagent are not the same.

In an embodiment of the above aspect, the first surface of the cartridge and the second surface of the cartridge are the same surface.

In another embodiment of the above aspect, the first surface of the cartridge and the second surface of the cartridge are different surfaces.

In some embodiments of any of the above aspect and embodiments, the at least one second analyte sensing reagent is bound to a second solid phase.

In some embodiments of any of the above aspect and embodiments, the at least one second analyte sensing reagent is bound to a second solid phase, the at least one second biomarker comprises NGAL, SDMA, CysB, fibroblast growth factor 23 (FGF-23), creatinine, blood urea nitrogen (BUN), clusterin, albumin, microalbumin, inosine, or an electrolyte selected from Na+, K+Ca2+, Mg2+, or phosphorus; wherein when the first biomarker comprises NGAL, the at least one second biomarker comprises SDMA, CysB, Na+, K+Ca2+, Mg2+, fibroblast growth factor 23 (FGF-23), creatinine, blood urea nitrogen (BUN), phosphorus, clusterin, albumin, microalbumin, or inosine;

when the first biomarker comprises SDMA, the at least one second biomarker comprises NGAL, CysB, Na+, K+Ca2+, Mg2+, fibroblast growth factor 23 (FGF-23), creatinine, blood urea nitrogen (BUN), phosphorus, clusterin, albumin, microalbumin, or inosine; and when the first biomarker comprises CysB, the at least one second biomarker comprises NGAL, SDMA, Na+, K+Ca2+, Mg2+, fibroblast growth factor 23 (FGF-23), creatinine, blood urea nitrogen (BUN), phosphorus, clusterin, albumin, microalbumin, or inosine.

In some embodiments of any of the above aspect and embodiments, the at least one second analyte sensing reagent is bound to a second solid phase, the first biomarker comprises NGAL, an NGAL analog, or an antibody specific for NGAL, or any combination thereof.

In some embodiments of any of the above aspect and embodiments, the at least one second analyte sensing reagent is bound to a second solid phase, the first biomarker comprises SDMA, an SDMA analog, or an antibody specific for SDMA, or any combination thereof.

In some embodiments of any of the above aspect and embodiments, the at least one second analyte sensing reagent is bound to a second solid phase, the first biomarker comprises CysB, a CysB analog, or an antibody specific for CysB, or any combination thereof.

In some embodiments of any of the above aspect and embodiments, the at least one second analyte sensing reagent is bound to a second solid phase, the device further comprises one or more additional solid phase having bound thereto one or more additional analyte sensing reagent or one or more additional antibody specific for one or more additional biomarker. In some further embodiments, the device further comprises N additional solid phases having bound thereto N additional analyte sensing reagents or N additional antibodies specific for N additional biomarkers. In yet some further embodiments, N is an integer selected from 1, 2, 3, 4, 5, 6, 7, or 8.

In some embodiments of any of the above aspect and embodiments, the at least one second analyte sensing reagent is bound to a second solid phase, the device further comprises a non-transitory computer-readable medium, having stored thereon program instructions that, upon execution by a controller, cause a controller to perform a set of operations comprising detecting a signal generated by the first analyte sensing reagent and the second analyte sensing reagent.

In another aspect, the disclosure provides a multiplex assay system or kit for determining kidney function and injury status in an animal subject, the system or kit comprising the device of any of the above aspect and embodiments or those otherwise described herein.

In some embodiments, the device, multiplex assay system, or kit according to any one of the above aspect and embodiments, finds use in methods for determining stable chronic kidney disease (CKD) in an animal subject.

In some embodiments, the device, multiplex assay system, or kit according to any one of the above aspect and embodiments, finds use in methods for determining progressive chronic kidney disease (CKD) in an animal subject.

In some embodiments, the device, multiplex assay system, or kit according to any one of the above aspect and embodiments, finds use in methods for early detection of chronic kidney disease (CKD) in an animal subject.

In some embodiments, the device, multiplex assay system, or kit according to any one of the above aspect and embodiments, finds use in methods for early staging of chronic kidney disease (CKD) in the animal subject.

In another aspect, the disclosure provides a method for determining kidney function and injury status in an animal subject, the method comprising the device, multiplex assay system or kit according to any one of the above aspects and embodiments, and further comprising measuring the concentration of at least a first and a second biomarker in a biological sample from the subject:
the first biomarker comprising:
   Neutrophil Gelatinase-Associated Lipocalin (NGAL), an NGAL analog, or an antibody specific for NGAL, or any combination thereof;
   Symmetrical Dimethylarginine (SDMA), an SDMA analog, or an antibody specific for SDMA, or any combination thereof; or
   Cystatin B (CysB), a CysB analog, or an antibody specific for CysB, or any combination thereof; and
the second biomarker comprising a sensing reagent or an antibody specific for the second biomarker; and
comparing the concentration of the first and second biomarkers to one or more standard values that are associated with normal kidney function and/or CKD or AKI in the animal subject, wherein the comparing indicates whether the subject has normal kidney function or kidney injury.

In another aspect, the disclosure provides a method for determining stable chronic kidney disease (CKD) in an animal subject, the method comprising the device, multiplex assay system or kit of any one of the above aspects and embodiments, and further comprising measuring the concentration of at least a first and a second biomarker in a biological sample from the subject:
the first biomarker comprising:
   Neutrophil Gelatinase-Associated Lipocalin (NGAL), an NGAL analog, or an antibody specific for NGAL, or any combination thereof;
   Symmetrical Dimethylarginine (SDMA), an SDMA analog, or an antibody specific for SDMA, or any combination thereof; or
   Cystatin B (CysB), a CysB analog, or an antibody specific for CysB, or any combination thereof; and
the second biomarker comprising a sensing reagent or an antibody specific for the second biomarker; and
comparing the concentration of the first and second biomarkers to one or more standard values that are associated with normal kidney function and/or stable CKD, wherein the comparing indicates whether the subject has stable CKD.

In another aspect, the disclosure provides a method for determining progressive chronic kidney disease (CKD) in an animal subject, the method comprising the device, multiplex assay system or kit of any one of the above aspect and embodiments, and further comprising measuring the concentration of at least a first and a second biomarker in a biological sample from the subject:
the first biomarker comprising:
   Neutrophil Gelatinase-Associated Lipocalin (NGAL), an NGAL analog, or an antibody specific for NGAL, or any combination thereof;
   Symmetrical Dimethylarginine (SDMA), an SDMA analog, or an antibody specific for SDMA, or any combination thereof; or
   Cystatin B (CysB), a CysB analog, or an antibody specific for CysB, or any combination thereof; and
the second biomarker comprising a sensing reagent or an antibody specific for the second biomarker; and
comparing the concentration of the first and second biomarkers to one or more standard values that are associated with normal kidney function and/or progressive CKD wherein the comparing indicates whether the subject has progressive CKD.

In another aspect, the disclosure provides a method for early detection of chronic kidney disease (CKD) in an animal subject, the method comprising the device, multiplex assay system or kit of any one of the above aspects and embodiments, and further comprising measuring the concentration of at least a first and a second biomarker in a biological sample from the subject:
the first biomarker comprising:
   Neutrophil Gelatinase-Associated Lipocalin (NGAL), an NGAL analog, or an antibody specific for NGAL, or any combination thereof;
   Symmetrical Dimethylarginine (SDMA), an SDMA analog, or an antibody specific for SDMA, or any combination thereof; or
   Cystatin B (CysB), a CysB analog, or an antibody specific for CysB, or any combination thereof; and
the second biomarker comprising a sensing reagent or an antibody specific for the second biomarker; and
comparing the concentration of the first and the second biomarker to one or more standard values that are associated with normal renal function in the animal subject wherein the comparing identifies early CKD in the animal subject.

In another aspect, the disclosure provides a method for early staging of chronic kidney disease (CKD) in an animal subject, the method comprising the device, multiplex assay system or kit of any one of the above aspects and embodiments, and further comprising measuring the concentration of at least a first and a second biomarker in a biological sample from the subject:
the first biomarker comprising:
   Neutrophil Gelatinase-Associated Lipocalin (NGAL), an NGAL analog, or an antibody specific for NGAL, or any combination thereof;
   Symmetrical Dimethylarginine (SDMA), an SDMA analog, or an antibody specific for SDMA, or any combination thereof; or
   Cystatin B (CysB), a CysB analog, or an antibody specific for CysB, or any combination thereof; and
the second biomarker comprising a sensing reagent or an antibody specific for the second biomarker; and
comparing the concentration of the first and the second biomarker to one or more standard values that are associated with normal renal function in the animal subject wherein the comparing identifies the stage of CKD in the subject.

In some embodiments of any of the above methods, the at least one second biomarker comprises NGAL, SDMA, CysB, fibroblast growth factor 23 (FGF-23), creatinine, blood urea nitrogen (BUN), clusterin, albumin, microalbumin, inosine, or an electrolyte selected from Na+, K+Ca2+, Mg2+, or phosphorus; wherein
when the first biomarker comprises NGAL, the at least one second biomarker comprises SDMA, CysB, Na+, K+Ca2+, Mg2+, fibroblast growth factor 23 (FGF-23), creatinine, blood urea nitrogen (BUN), phosphorus, clusterin, albumin, microalbumin, or inosine;
when the first biomarker comprises SDMA, the at least one second biomarker comprises NGAL, CysB, Na+, K+Ca2+, Mg2+, fibroblast growth factor 23 (FGF-23), creatinine, blood urea nitrogen (BUN), phosphorus, clusterin, albumin, microalbumin, or inosine; and
when the first biomarker comprises CysB, the at least one second biomarker comprises NGAL, SDMA, Na+, K+Ca2+, Mg2+, fibroblast growth factor 23 (FGF-23), creatinine, blood urea nitrogen (BUN), phosphorus, clusterin, albumin, microalbumin, or inosine.

Other aspects and embodiments will be apparent to those of skill in the art in light of the detailed description that follows.

### BRIEF DESCRIPTION OF THE DRAWINGS

The accompanying drawings, which are included to provide a further understanding of the disclosure, are incorporated in and constitute a part of this specification, illustrate embodiments of the disclosure and together with the detailed description serve to explain the principles of the invention. No attempt is made to show structural details of the invention in more detail than may be necessary for a fundamental understanding of the invention and various ways in which it may be practiced.

FIG. 1 depicts a diagrammatic illustration of an assay system including a device having a waveguide with an integrated lens, illumination, and imaging system, in accordance with an example embodiment of the disclosure.

FIG. 2 depicts a top view of one of the devices (e.g., a rotor or disc) in accordance with some exemplary aspects and embodiments of the disclosure.

FIGS. 3a-3e depict schematic illustrations of a device (e.g., microfluidic assay system) in accordance with certain example embodiments of the disclosure, where a reaction section can be inclined relative to a horizontal plane (3e).

FIG. 4 depicts a side view of one of the devices (e.g., a cartridge) in accordance with some exemplary aspects and embodiments of the disclosure.

### DETAILED DESCRIPTION

In its various aspects, the disclosure relates to devices, multiplex point-of-care diagnostic assays, methods, kits, and compositions, that comprise first and second biomarkers of kidney function and/or injury in a biological sample from a subject. In embodiments of these aspects, the disclosure provides for devices and multiplex assay systems that are useful in the determination, diagnosis, staging, progression and prognosis of kidney function, kidney injury status, and/or kidney disease.

In a general aspect, the disclosure provides a device for determining kidney function and injury status in an animal subject, the device configured to receive a biological sample from the animal subject and comprising a solid phase having bound thereto at least a first analyte sensing reagent and a second analyte sensing reagent:
the first analyte sensing reagent comprising a sensing reagent or an antibody specific for a first biomarker comprising:
   Neutrophil Gelatinase-Associated Lipocalin (NGAL), an NGAL analog, or an antibody specific for NGAL, or any combination thereof;
   Symmetrical Dimethylarginine (SDMA), an SDMA analog, or an antibody specific for SDMA, or any combination thereof; or
   Cystatin B (CysB), a CysB analog, or an antibody specific for CysB, or any combination thereof;
the second analyte sensing reagent comprising a sensing reagent or an antibody specific for a second biomarker; and
wherein the first and second biomarker are not the same.

In some embodiments, the disclosure relates to a method for determining kidney function, kidney injury status, and/or kidney disease staging utilizing a multiplex point-of-care diagnostic that comprises detecting at least one of NGAL, SDMA, and CysB, in combination with one or more biomarkers in a biological sample from an animal such as a mammal (e.g., feline, canine, human, etc.). Various sensing reagents, antibodies, biomarkers, methods, reagents and techniques can be used in accordance with the disclosure (see, e.g., U.S. Pat. Nos. 11,035,861, 10,436,797, and 11,933,792, all of which are incorporated herein by reference in their entireties). The disclosed devices, multiplex assays, kits, and compositions can be useful in the identification, diagnosis, and treatment of kidney disease or dysfunction including, for example, identifying, distinguishing, and/or staging chronic kidney disease, including distinguishing progressive CKD from stable CKD.

### Definitions

CysB is Cystatin B.

BUN is blood urea nitrogen.

NGAL is neutrophil gelatinase-associated lipocalin.

SDMA is symmetrical dimethylarginine.

As used herein, an animal or mammalian “patient” or “subject” can mean a human or non-human animal including canine, feline, bovine, porcine, or equine.

The term “sample,” “test sample,” “patient sample,” or “subject sample” as used herein includes but is not limited to a blood, serum, plasma, saliva, plaque, crevicular fluid, gingival biopsy, tongue swab, or urine sample obtained from a subject. The test sample can be untreated, precipitated, fractionated, separated, diluted, concentrated, or purified.

The term “analog,” as used herein, generally refers to a compound in which one or more individual atoms have been replaced with a different atom(s) or with a different functional group(s). For example, an analog may be a modified form of the analyte which can compete with the analyte for a receptor, the modification providing a means to join the analyte to another moiety, such as a label or solid support. The analyte analog can bind to an antibody in a manner similar to the analyte.

### Devices and Assay Platforms

The disclosure provides, in various aspects and embodiments, devices, multiplex assays, kits, and methods for performing assays to determine the presence or quantity of one or a combination of selected specific analyte(s) of interest in a sample such as, for example, a biological sample (e.g., tissue or fluid).

In some aspects, the disclosure provides a device that can analyze (e.g., assay) a measured amount of sample wherein the device comprises at least two separate and distinct flow paths which are initiated simultaneously with a single user activation step and one or more reaction zone(s). In embodiments of these aspects, the paths can be timed for the sequential delivery of assay reagents to the reaction zone, followed by wash or substrate and wash reagents to that reaction zone or another reaction zone(s). In some embodiments, the devices and methods provide for the controlled, self-delivery of reagents with no timed steps, and minimal user intervention, in most instances a single activation step.

In other aspects, the disclosure provides devices and methods comprising a centrifugal force that can be used to direct fluid sample through radial flow paths such that the fluid sample contacts reagents and immobilized binding partners positioned within the flow paths. In embodiments of these aspects, a detection device employing radial flow paths avoids any need to incorporate or use the overlapping porous surfaces of conventional lateral flow devices to achieve proper sample flow through the device. In embodiments, such devices can contain multiple radial flow paths and thus the presence of multiple analytes can be detected simultaneously in a single sample. Accordingly, these aspects and embodiments provide an analyte detection system in a rotor or disc format that allows for the detection of multiple analytes in a biological sample.

In a further embodiment, the detection system comprises a centrifugal force, a sample port and a surface, wherein the surface comprises at least one channel, said channel containing an immobilized capture ligand capable of specifically binding to an analyte in a sample, wherein the sample port is in fluid communication with said at least one channel, and wherein the centrifugal force is operably connected to the sample port so that when in operation it causes a sample deposited in the sample port to move through the at least one channel and be in fluid contact with the capture ligand. In some embodiments, the one or more channels can be part of a flow path allowing the sample to flow radially outward when a centrifugal force is applied to the system. In other embodiments, the one or more channels can be part of a flow path allowing the sample to flow along a circular path when a centrifugal force is applied to the system. In another embodiment, the surface further contains at least one absorbing entity located downstream from the capture ligand.

In some further embodiments, the channel further contains a conjugate capable of binding to the analyte in the sample to form a complex, wherein the complex is captured by the capture ligand. Conjugates present in the one or more channels of the surface can comprise a binding partner conjugated to a detectable entity, wherein the binding partner is capable at specifically binding to a target analyte in a sample. In some embodiments, the binding partner is an antibody or antigen. The detectable entity can be a metal particle (e.g. metal nano-particle or metal nanoshell), fluorescent molecule, colored latex particle, or an enzyme. In one embodiment, the detectable entity is a gold nanoparticle.

In another embodiment, the channel comprises a first flow path and a second flow path, wherein said first and second flow paths are positioned in different planes, and wherein said first and second flow paths are in fluid communication. The first flow path can comprise an immobilized capture ligand and a conjugate capable of binding to an analyte in a sample to form a complex that can be captured by the capture ligand. The second flow path can comprise a substrate region, which comprises a substrate entity capable of interacting with the detectable entity of the conjugate to produce or amplify a detectable signal. In one embodiment, the first flow path provides a faster flow through than the second flow path when a centrifugal force is applied to the channel

In certain further embodiments, the surface of the detection system comprises a plurality of channels (e.g., two or more channels), wherein each said channel comprises an immobilized capture ligand capable of specifically binding to an analyte in a sample. In one particular embodiment, each channel further comprises a conjugate capable of binding to an analyte the sample to form a complex and wherein the complex is captured by a capture ligand. In some embodiments, each conjugate specifically binds to a different target analyte in a sample. In one embodiment, each of the channels comprises a first and second flow path, wherein said first and second flow paths are located in different planes and are in fluid communication.

In another embodiment, the sample port of the detection system comprises one or more conjugates capable of binding to an analyte in the sample to form a complex, wherein the complex is captured by a capture ligand. The conjugates can bind different analytes the sample and may, in some embodiments, contain different detectable entities. In certain embodiments, the surface of the detection system contains a channel, wherein the channel comprises a first capture ligand and a second capture ligand, wherein the first capture ligand is located upstream from the second capture ligand. In one embodiment, the first capture ligand specifically binds a different analyte than the second capture ligand.

In some embodiments, the one or more channels of the detection system further contain a positive or negative control entity. The control entity can comprise a control binding partner that binds to the conjugate (e.g. binding partner or detectable entity). A detection signal from the control entity can indicate proper fluid flow through the detection system.

In other aspects, the disclosure provides a kit comprising an analyte detection system as described herein and instructions for using the system to detect one or more target analytes in a sample. The detection systems are adapted for use with a centrifugal force and in some embodiments, can be used with conventional centrifuges with appropriate attachments. The kit can further comprise means for collecting samples and buffers for extracting samples from solid substances.

In other aspects, the disclosure provides a method for detecting an analyte in a sample. In one embodiment, the method comprises adding the sample to the sample port of an analyte detection system of the invention, applying a centrifugal force to the system, and detecting the binding of the analyte to the capture ligand. The sample can be a biological sample isolated from a human or animal subject in some embodiments, multiple (e.g., two or more) analytes are detected from a single sample simultaneously.

In other aspects, the systems, devices, kits, and methods comprise a microfluidic cartridge suitable for a fast and accurate temperature adjustment of a sample contained therein. In embodiments, the microfluidic cartridge can comprise a reaction section where a sample in the reaction section can be incubated at a desired, adjustable, controlled, and accurate temperature. In embodiments, the reaction section can be subjected to a temperature regulation according to a predetermined temperature plan.

In these aspects and embodiments, the systems, devices, kits, and methods provide quantitative or qualitative heat sensitive assays, such as determination of a target component in a biological sample, e.g., where the determination includes a temperature sensitive assay, such as a biochemical assay.

In these aspects and embodiments, the disclosure provides a microfluidic assay system comprising a microfluidic cartridge and an associated microfluidic operator system. As may be suitable or desired, the microfluidic cartridge can be disposable while the microfluidic operator system can be repeatedly used together with microfluidic cartridges of the same design and size or with microfluidic cartridge with different shapes and sizes. As such, the microfluidic operator system can optionally be adjustable such that it may be used with microfluidic cartridge of different sizes or shapes.

In accordance with some of these embodiments, a microfluidic cartridge comprises a base part, having a first face and a second opposite face and with a recess in the first face and a foil fixed to the base part for covering the recess and providing a microfluidic cartridge foil face which is the face of the foil facing away from the base part. In embodiments, the base part is rigid. In embodiments, the foil may be fixed to the base part by any means, such as by being welded, molded, or glued. The foil is fixed to the base part such that the recess and the foil form a flow channel and a sink. The flow channel has a length and comprises a reaction section and an upstream end and a downstream end. In an embodiment the upstream end is on one side of the reaction section and the downstream end is on an opposite side of the reaction section. It should be understood that the flow channel may have two or more reaction sections. In some embodiments, the flow channel can be branched such that it comprises more than one flow path.

The sink is in fluid communication with the flow channel downstream to the reaction section. The microfluidic cartridge comprises an inlet opening into the flow channel upstream to the reaction section.

In these aspects and embodiments, the operator system suitably comprises a piston, a temperature regulating element and an actuator positioned such that the foil face of the microfluidic cartridge can be positioned in contact with the operative system with the reaction section in close proximity to the temperature regulating element while the actuator is associated to the sink section to depress the foil covering the sink section and the piston is associated to the flow channel at an upstream valve section to depress the foil to close off the flow channel upstream to the reaction section.

In accordance with these embodiments, the microfluidic assay system can provide for effective temperature regulation of a sample within the reaction section of the microfluidic cartridge. Suitably, the foil can be relatively thin which allows for rapid heat transfer through the foil. The arrangement of the sink and the upstream valve section can provide for the ability to raise the pressure within the reaction section such that the foil covering the reaction section does not deflect into the recess of the base part. The arrangement of these elements provide for the intimate contact of the foil face and temperature regulating element which allows for accurate temperature adjustment of a sample within the reaction section

In some other aspects and embodiments, the disclosure provides a system comprising a cartridge comprising a fluidic channel, a waveguide, and a capture spot disposed on the waveguide and within the fluidic channel. In some embodiments, the system further includes a force field generator and an imaging system. The first and/or second analyte sensing reagents can be responsive to a force field, and are capable of generating a signal. The force field allows manipulation of at least one of the first and/or second analyte sensing reagents such that the signal is indicative of presence of the target analyte within the sample. In further embodiments, the first and/or second analyte sensing reagents comprise magnetic particles, and the force field generator is a magnet. For example, the magnetic particles are polystyrene microspheres including a magnetic component (e.g., core and/or coating). In yet further embodiments, at least one of the first and/or second analyte sensing reagents comprise luminescent particles.

In still further embodiments, the multiple-particle complex exhibits directional signal enhancement.

In a further embodiment, the system also includes an excitation source for providing excitation energy so as to illuminate at least a portion of the fluidic channel. The second set of particles are fluorescent particles configured for generating a fluorescent signal when the excitation energy is incident thereon.

In a yet further embodiment, the waveguide is a planar waveguide such that the excitation energy is directed into the portion of the fluidic channel at least in part by total internal reflection through the planar waveguide.

In a still further embodiment, the cartridge includes a plurality of capture spots disposed on the waveguide and within the fluidic channel.

In a further embodiment, the imaging system includes an image sensor selected from a group consisting of a charge-coupled device (“CCD”) and a complementary metal-oxide-semiconductor (“CMOS”) sensor.

In some embodiments, the device in accordance with the disclosure can comprise a cartridge comprising a sample reservoir, a solution reservoir, a paramagnetic, bar-coded bead reservoir, an assay component reservoir, a testing reservoir, and a waste reservoir, all of which reside on a dielectric cartridge surface. In this example embodiment the device comprises a plurality of electrodes and at least one magnet that are disposed along various portions of the dielectric cartridge surface. The plurality of electrodes can be configured to facilitate transportation of a biological sample (e.g., fluid droplet) containing at least one paramagnetic, bar-coded bead along dielectric cartridge surface, and the at least one magnet immobilizes the fluid droplet and/or components thereof (e.g., at least one paramagnetic, bar-coded bead). In accordance with this embodiment, the term “dielectric cartridge surface” includes cartridge surfaces below any reservoir such as, for example a sample reservoir, a solution reservoir, a paramagnetic, bar-coded bead reservoir, an assay component reservoir, a testing reservoir, and a waste reservoir, as well as any path that connects any one or all of such components.

In some embodiments, the cartridge and/or any components thereof may interact with a computing device, such as those known in the art or as described briefly herein. A computing device can be implemented as a controller, and a user of the controller can use the controller to program and/or control the cartridge and/or any components thereof. The cartridge and/or any components thereof may be communicably coupled with a controller, such as the computing device, and may communicate with the controller by way of a wired connection, a wireless connection, or a combination thereof. Further, a controller may be configured to control various aspects of the cartridge and testing protocols (e.g., assays) utilizing the cartridge and/or any components thereof. In accordance with this embodiment, various cartridge components and arrangements of these components can be implemented (e.g., different shapes, amounts, and/or types of beads, particles, and/or components).

In some embodiments, the device can include a lateral flow assay device. Lateral flow devices are known (see, e.g., U.S. Published Patent Application Nos. 2005/0175992 and 2007/0059682, the contents of which are incorporated herein by reference). These and any other lateral flow devices known in the art can be used with the systems and methods described herein. In some non-limiting embodiments, a lateral flow immunoassay device in accordance with the disclosure includes a sample-transporting liquid, which can be a buffer, and a chromatographic test strip containing one or several materials or membranes with capillary properties through which sample flows. Some example materials and membranes for the test strip include, but are not limited to, Polyethylene terephthalate (PET) fibers, such as Dacron® fibers, nitrocellulose, polyester, nylon, cellulose acetate, polypropylene, glass fibers, and combinations of these materials and their backings. In some embodiments, treatment of a sample (e.g., lyse the cells in the sample or treat the sample in any way prior to applying it to the test strip) may be needed or preferable.

In some embodiments, the device can comprise a chromatographic test strip, to determine if one or more biomarkers are present. In such embodiments, a sample is collected, and transferred to the chromatographic test strip and the test strip includes a reagent zone. The reagent zone can include at least one first reagent specific to a biomarker such that, when the biomarker is present in the sample contacts the first reagent, a first labeled complex forms. The reagent zone also preferably includes at least one second reagent specific to a second biomarker such that, when the second biomarker present in the sample contacts the second reagent, a second labeled complex forms. A detection zone includes both a first biomarker binding partner which binds to the first labeled complex and a second biomarker binding partner which binds to the second labeled complex. The sample is then analyzed for the presence of the first and second markers. In such embodiments, the device can include a sample application zone. In some embodiments, the presence of one or more biomarkers is indicated by a test line visible to the naked eye. The presence of the first biomarker may be indicated by a first test line while the presence of the second biomarker can be indicated by a second test line. In some embodiments, the first test line displays a first color when positive and the second test line displays a second color different from the first color when positive. In embodiments where both the first test line and the second test line are located in the same space on the sample analysis device, a third color can be formed when both the first test line and the second test line are positive. In other embodiments, the two test lines are spatially separate from each other on the device.

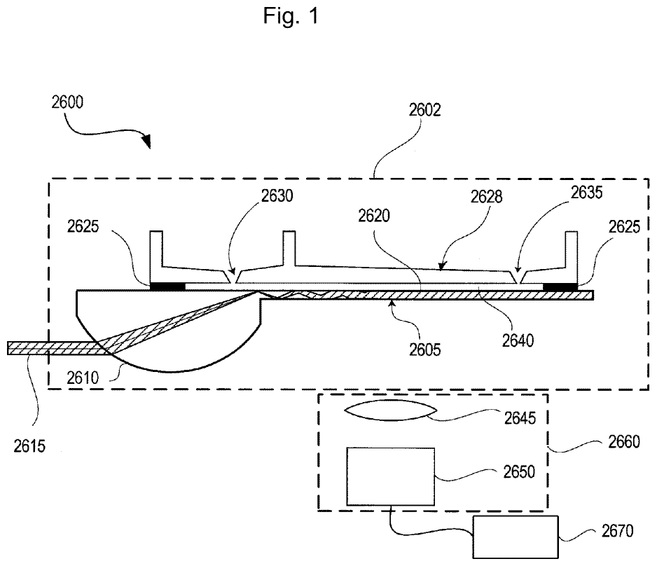

Exemplary embodiments of the devices and systems illustrated in FIGS. 1-4, are provided merely for purposes of illustration and description. Briefly, FIG. 1 depicts a representation of an exemplary disposable assay cartridge and a multiplexed fluorescence assay, in accordance with an embodiment. In this example, a protein microarray is printed to a plastic planar waveguide which is bonded to a plastic upper component to define a flow channel. Fluids, such as sample, wash, and detect reagents may be introduced via an inlet port. The assay surface may be illuminated by an evanescent field generated down the length of the multi-mode waveguide. The array is imaged in a single field of view through the plane of the waveguide.

At the TIR interface, an evanescent field is generated that decays exponentially into the aqueous medium. The decay length of the evanescent field is on the order of a hundred nanometers for visible light. For fluorescence assay applications, the advantage is localization of the illumination source precisely at the solid-liquid assay interface, limiting negative effects such as the bulk solution, line-of-sight, light scattering.

The cartridge is based on a thick (“1 mm), multi-mode planar waveguide fabricated by injection molding of a low auto-fluorescence plastic (e.g., cyclic olefin polymer). One advantage of such a cartridge configuration can be an incorporation of a coupling lens into a molded waveguide. This lens design overcomes fundamental challenge of reproducible light coupling to the waveguide in prior designs. The lens design creates a diverging beam such that modes mix down the length of the waveguide, eventually creating a spatially uniform illumination field along the axial length of the waveguide.

The plastic waveguides are activated with a surface chemistry treatment to render them amine-reactive. Details of the surface activation are similar to methods described in the literature.

A protein array can be printed to the activated surface of the planar waveguide prior to assembly into the cartridge. Such arrays may be printed with a commercial arrayer, such as Bio-Dot AD3200 robotic arrayer equipped with Bio-Jet print head dispensing 28 nanoliter droplets. Resulting reaction site diameters can be approximately 0.5 mm, and the arrays can be printed on a grid with 1.25 mm centers. The length of the 30 feature (i.e., 2 rows by 15 columns in the present example) array can be approximately 17.5 mm. After printing, the waveguide arrays can be rinsed with a protein-based blocking agent, spin-dried, and then coated with a sugar-based stabilizer for storage.

Printed waveguides can be assembled into an injection molded cartridge to form a 2 to 5 mm-wide fluidic channel with a volume of approximately 30 microliters. The cartridge inlet port can provides a reservoir for introduction of assay fluids. The exit port can provides a fluidic contact to an absorbent pad that can serve as a waste reservoir. The cartridge can be configured to provide reproducible passive fluid flow, driven by a combination of capillary action and hydrostatic pressure, as is generally known in the art. All fluids stay on board the cartridge upon completion of the assay procedure, thus minimizing biohazard. In this way, a combination of printed antigens, controls, and a sample placed in the liquid channel may be used to perform an assay.

FIG. 1 illustrates a cross-sectional view of an assay system 2600, including a cartridge 2602. Cartridge 2602 includes a planar waveguide 2605 with an integrated lens 2610 suitable for use with the labeled antigen assay. An illumination beam 2615 is inserted into planar waveguide 2605 through integrated lens 2610. Illumination beam 2615 may be provided, for example, by a laser with an appropriate wavelength to excite fluorescent labels at an assay surface 2620. Other appropriate forms of illumination, either collimated or uncollimated, may also be used with assay system 2600. Integrated lens 2610 is configured to cooperate with planar waveguide 2605 such that illumination beam 2615, so inserted, is guided through planar waveguide 2605 and may illuminate assay surface 2620 by evanescent light coupling. Assay surface 2620, an upper component 2628, which includes an inlet port 2630 and an output port 2635, cooperate to define a fluidic sample chamber 2640. Assay surface 2620 and upper element 2628 can be bonded via a channel-defining adhesive gasket 2625 or via direct bonding methods such as laser welding, ultrasonic welding, or solvent bonding. Appropriate chemical compounds (such as a printed antigen) are bound to assay surface 2620 such that when a biological sample and labeled detect reagent are added to the fluidic sample chamber 2640, a target analyte, if present, forms a sandwich between its specific labeled detect reagent and its specific chemical compound immobilized on assay surface 2620. If the specific complex is formed at assay surface 2620, fluorescence signal at the immobilized compound location is indicative of the presence of the target analyte within the biological sample. As an example, collection and filtering optics 2645 may be used to capture the fluorescence signal from assay surface 2620. A signal corresponding to the fluorescence so captured may then be directed to an imaging device 2650, such as a CCD or CMOS sensor.

In a further embodiment, assay system 2600 may be used for rapid, simple detection of multiple target antibodies in a single biological sample. Multiple different antigens may be immobilized at reaction sites on the assay surface, such as in stripes or spots in an array format using printing technology, thereby creating a spatially-localized set of parallel assay locations. The combination of a biological sample, labeled antibody against human IgG, and immobilized antigens on assay surface 2620 may lead to the formation of multiple physically separated antigen-antibody complexes on the assay surface. Illumination of assay surface 2620 results in spatially-localized fluorescence signal that may be read with a detection system 2660 including collection and filtering optics 2645, imaging device 2650, and computer 2670. Computer 2670 may be integrated into the detection system instrument (e.g., single board computer). Alternatively, computer 2670 could be an external device, such as a peripheral device.

Additional features and alterations can be incorporated into the system (e.g., cartidge and devices) of FIG. 1 as described, for example, in U.S. Pat. No. 8,586,347 which is incorporated herein by reference in its entirety

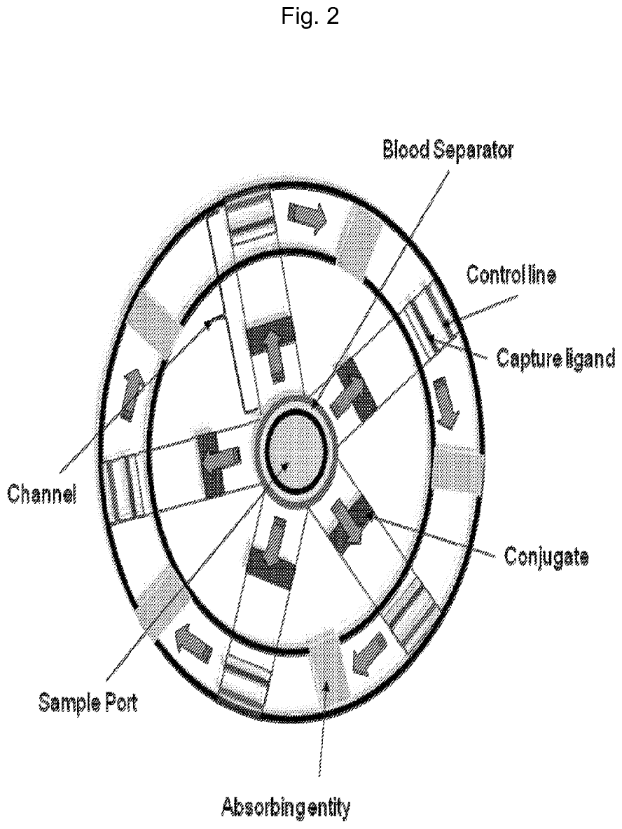

FIG. 2 illustrates an embodiment comprising a surface (e.g. rotor base or disc) that contains a plurality of channels that are in fluid communication with a sample port. Each channel contains a conjugate comprised of a binding partner conjugated to a detectable entity that can specifically bind to a target analyte present in the sample. At the peripheral edge of each channel is a capture ligand capable of binding the analyte-conjugate complex. The peripheral edge of each channel can optionally contain a control line that indicates sufficient fluid flow through the system. When a centrifugal force is applied to the surface and sample port, a fluid sample deposited in the sample port flows radially through the channels to the periphery of the surface (Hue arrows), and excess fluid is absorbed by absorbent material (absorbing entity) positioned downstream of each capture ligand. The surface can optionally contain a blood separator material, which allows plasma from a blood sample to pass into the system while retaining cellular material in the sample port. Additional features and alterations can be incorporated into the device of FIG. 2 as described, for example, in U.S. Pat. No. 10,969,385 which is incorporated herein by reference in its entirety.

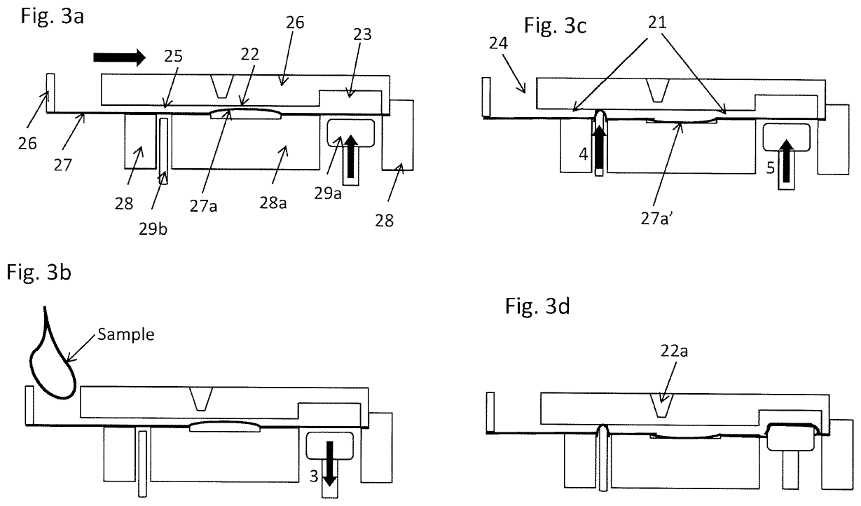

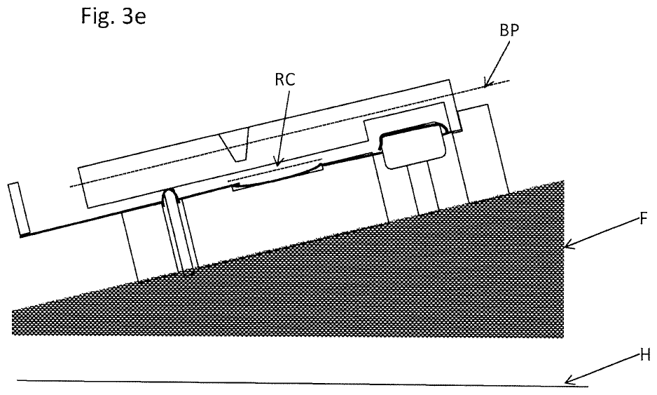

As illustrated in FIGS. 3a, 3b, 3c and 3d some example embodiments of the disclosure can comprise microfluidic assay system, which may operate in accordance with the depictions. In the Figures, a microfluidic assay system can comprise a microfluidic cartridge and an associated microfluidic operator system. The microfluidic cartridge can comprise a base part 26, having a first face and a second opposite face and with a recess in the first face and a foil 27 fixed to the base part 26 for covering the recess. The base part 26 with the recess and the foil 27 form a flow channel 21 and a sink 23. The microfluidic cartridge can comprise an inlet opening 24 which is formed by an orifice in the base part 26 as described herein or generally known in the art.

By providing the orifice sufficiently large, such that the edge of the base part in the periphery of the orifice and the foil 17 covering the orifice provides a cavity sufficiently large for a drop of the sample, the risk of spilling sample is reduced.

The flow channel 21 can comprise an upstream valve section 25 and a reaction section 22.

The operator system can comprise a supporting frame 28, a piston 29 b, a temperature regulating element 28 a and an actuator 29 a positioned such that the foil face of the microfluidic cartridge can be positioned in contact with the operative system with the reaction section 22 in close proximity to the temperature regulating element 28 a while an actuator 29 a is associated to the sink section 23 to depress the foil 27 covering the sink section 23 and the piston 29 b is associated to the flow channel 21 at the upstream valve section 25 to depress the foil 27 to close off the flow channel 21 upstream to the reaction section 22.

In FIG. 3a the microfluidic cartridge is positioned in contact with the microfluidic operator system. As seen the reaction section 22 is positioned in close proximity to the temperature regulating element 28 a, the actuator 29 a is associated to the sink section 23 to depress the foil 27 covering the sink section 23 and the piston 29 b is associated to the flow channel 21 at the upstream valve section 25 to depress the foil 27 to close off the flow channel 21 upstream to the reaction section 22.

As it can be observed, the foil 27 a covering the reaction section has a tendency to deflect towards the base part to decrease the volume of the reaction section. This effect has shown to be increased when the reaction section comprises fluid, unless a pressure is applied.

As indicated at the actuator 29 a, the actuator is activated to depress the foil 27 covering the sink 23 to thereby press air out of the flow channel 21 via the inlet 24. Thereafter as shown in FIG. 3b, a drop of sample is applied to the inlet 24 and the actuator is released, whereby the sample is sucked into the flow channel 21 and the reaction section 22. In FIGS. 3c and 3d the sample is not shown but it should be interpreted that the sample is in the microfluidic cartridge.

In FIG. 3c the piston 29 b is activated to close off the upstream valve section 25. Thereafter the actuator 29 a is activated to depress the foil 27 covering the sink 23 to thereby raise the pressure in the reaction section slightly such that the foil 27 a′ covering the reaction section 22 is no longer depressed towards the base part 26, but rather is deflected away from the base part 26 i.e. it is slightly bugled.

FIG. 3d shows the actuator 29 a as it is depressing the foil 27 covering the sink 23.

The microfluidic cartridge comprises a withdrawing depression 22 a which may be used to remove a sample from the reaction section 22. A syringe with a needle may be used to puncturing the thin wall into the reaction section 22 at the withdrawing depression 22 a.

FIG. 3e corresponds to FIG. 3d where the operator system comprises a foundation F for ensuring that at least the reaction section is inclined relative to a horizontal plane. It can be seen that the center axis RC of the reaction center is inclined relative to a horizontal plane H and also the plane PB of the base part is inclined relative to the horizontal plane H. Due to the inclined position any bubbles formed in the reaction chamber e.g. caused by the temperature regulation will migrate to the sink section and thus such bubbles will not deteriorate the optical read out. Additional features and alterations can be incorporated into the device of FIGS. 3a-3e as described, for example, in U.S. Pat. No. 11,400,449 which is incorporated herein by reference in its entirety.

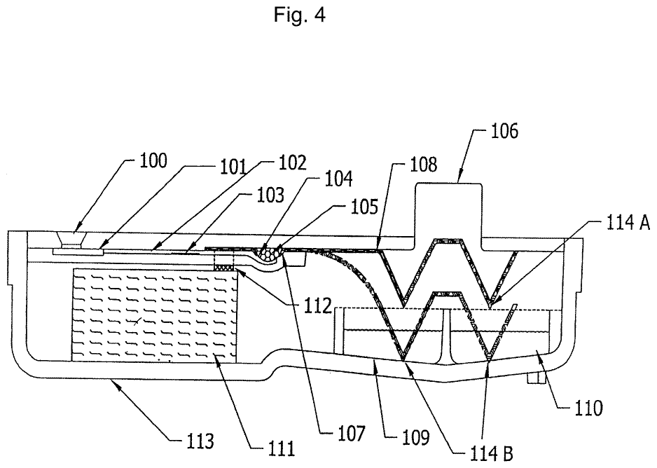

FIG. 4 shows a side view of an embodiment of a device. In this embodiment, the devices have thicknesses of about 1-30 mm, lengths of about 3-15 cm, widths of about 1-10 cm. Device size is determined by a large number of aesthetic, ergonomic, and performance factors; for example width is affected by number of sample delivery channels 102, length by time to result desired, and thickness is affected by choice of reservoir and absorbent block materials. The device depicted in FIG. 1 shows some of the general features of the devices falling within the scope of the disclosure. The device comprises various elements: a sample entry port 100, pre-filter 101, one or more sample delivery channel(s) 102, socklet 104, solid phase 105, second fluid path material 108, wash reservoir 109, substrate reservoir 110. Both the wash reservoir 109 and the substrate reservoir 110 have lances 114A & B, which can serve as release mechanisms and wicks.

Referring further to FIG. 4, the sample is added to the sample entry port 100 and flows through a pre-filter 101. The sample entry port 100 can be any opening in the device housing for receiving sample and transferring it to the desired location for the start of the assay. Multiple sample delivery channels 102 may also be incorporated into the device. For example, if one sample is being tested for the presence of multiple analytes, once the sample is applied, the device is designed such that equal aliquots are deposited in multiple sample delivery channels. Devices with multiple sample delivery channels will be discussed in more detail below. Once the sample goes through the pre-filter 101, the sample flows into the hydrophilic sample delivery channel 102 where dried conjugate soluble binding reagents 103 are located. When the liquid sample enters sample delivery channel 102 the conjugate soluble binding reagents 103 are dissolved into the sample solution and mobilized. Mixing the sample and conjugate allows the binding reactions to begin. Depending on the assay format complexes may form between binding reagents and analyte in the sample. These binding interactions continue to form while reagents continue to flow through the sample delivery channel 102.

Once the sample/conjugate mixture reaches the distal end of sample delivery channel 102, the sample/conjugate mixture flows to the solid phase 105. This embodiment, as illustrate, incorporates a socklet 104, a hydrophilic mesh which holds a particulate solid phase material. The embodiment may alternatively omit a socklet, for example when non-particulate solid phase materials are chosen. Additional features and alterations can be incorporated into the device of FIG. 4 as described, for example, in U.S. Pat. No. 6,436,722 which is incorporated herein by reference in its entirety.

The devices and methods described herein may be used for qualitative, semi-quantitative and quantitative determinations of one or multiple analytes in a single test format. The devices and methods may be practiced with ELISA, sol particle and other assay formats, and are particularly suitable for simultaneous multiple analyte assays. Assays of this invention are in a unit dose format, stable, capable of room temperature storage, reliable, easy to manufacture and use, and available for a low cost per test. They have fully integrated packaging for both liquid and dried reagents. In addition, the devices can be self-timing for the delivery of reagents so there is minimal operator involvement.

Biomarkers: Neutrophil Gelatinase-Associated Lipocalin (NGAL), Symmetrical Dimethylarginine (SDMA), and Cystatin B (CysB) in identifying status of kidney function and injury

In various aspects, the disclosure relates to the use and/or detection of a combination of biomarkers (e.g., at least one of NGAL, SDMA, and CysB) in a sample from an animal subject that has or is suspected of having impaired kidney function (e.g., chronic kidney disease (CKD)) and/or kidney injury (e.g., acute kidney injury (AKI)), wherein the amount of NGAL detected in the sample identifies the subject as having, or not having, CKD or AKI. In further aspects the disclosure relates to the use of a multiplex point-of-care assay for identifying the status of kidney function and/or injury in a subject, wherein the multiplex point-of-care assay comprises detecting in a sample from the subject at least two biomarkers comprising at least one of NGAL, SDMA, and CysB and at least one other biomarker associated with kidney function and/or injury, and wherein the detected amount of the at least two biomarkers in the sample identifies the status of kidney function and/or injury in the subject.

Chronic kidney disease (CKD) can be staged (e.g., Stages I-IV) and characterized as stable or progressive is defined as presence of structural or functional abnormalities in the kidney for greater than 3 months. In its early stages, CKD may be caused by one or more relatively minor insults to the kidneys that are not associated with any acute clinical symptoms or any typical elevated levels of a diagnostic biomarker. Eventually, CKD can transition from stable disease (stable CKD) to progressive which exhibits common clinical symptoms such as increased water consumption, frequent urination, diminished appetite, weight loss and muscle atrophy, and can eventually progress to fibrosis and degeneration of the kidney parenchyma. CKD is irreversible and tends to be progressive in nature. CKD is typically diagnosed based on changes in functional markers, such as serum creatinine and symmetric dimethylarginine (SDMA) concentration, which are also used to monitor the disease progression. Yet these functional markers can be insensitive in early CKD due to the nonlinear relationship with glomerular filtration rate (GFR) and due to a relatively wide reference range for both creatinine and SDMA. Markers of active ongoing injury (Acute Kidney Injury; AKI) can also be increased in some dogs with CKD, indicating that active ongoing injury is also present in dogs with CKD, even when the disease seems to be stable based on functional markers.

Generally, by the time clinical symptoms of CKD develop, irreparable kidney damage has occurred. Early detection permits earlier treatment and in turn slows disease progression. As such, early detection can be crucial for improving life span and quality of life. Therefore, CKD and AKI may not be completely separate processes and may share some common characteristics.

Although CKD tends to be progressive in all dogs, the degree of progression varies substantially among dogs. Such differences in progression can be observed in the rate of change, or slope, of functional renal biomarkers (e.g. creatinine, SDMA). When assessing progression of CKD functional renal biomarker values can be converted to their reciprocals (“inverse biomarker”), from which slopes (“inverse slopes”) can be calculated. Several risk factors for CKD progression have been identified, including the degree of renal proteinuria as reflected by urine protein: creatinine (UPC) ratio, hypertension, and hyperphosphatemia. It is possible that the degree of active injury in animals (e.g., cats and/or dogs) with CKD might serve as a surrogate marker for the progression rate, regardless of the underlying cause.

Stage 1 chronic kidney disease (CKD) based on IRIS guidelines is typically associated with being more stable than CKD in advanced stages. However, some IRIS Stage 1 CKD dogs, specifically those with progressive CKD, may have active, ongoing injury as reflected by elevated NGAL values throughout the follow-up period. Further, NGAL can provide an earlier indicator of such injury compared to traditional biomarkers.

CKD in dogs and cats is a multifactorial disorder with multiple implicated etiologies. In the majority of animals with CKD, an underlying cause cannot be identified or eliminated. In the absence of an identifiable cause that can be eliminated, therapy is focused on dietary intervention, which has been shown to slow down progression rate, and controlling risk factors for rapid progression (e.g., hypertension, proteinuria). Once the disease has progressed to more advanced stages, treatment transitions to a more symptomatic focus. Therefore, a major therapeutic goal is to identify the disease early in its course and to slow down the progression rate before substantial irrecoverable damage occurs.

Diagnosis of stable or early stage CKD (stage 1) in dogs and cats is challenging and additional methods of detecting small or subclinical kidney insults and injury are needed. Currently the diagnosis of early stage or stable CKD cannot be established based solely on functional markers. Therefore, early CKD diagnosis is based on presence of persistently non-concentrated urine (after exclusion of all other potential causes), presence of renal proteinuria, and ultrasonographic changes (which are often subtle and inconclusive at this stage of the disease). Many of these techniques require multiple visits to the veterinarian. Other diagnostics include measuring glomerular filtration rate (GFR) and kidney biopsy which are burdensome and invasive, and neither of which are routinely used in the clinic. Progressive CKD in animal subjects can have elevated levels of at least one of NGAL, SDMA, and CysB at first visit compared to animal subjects with stable CKD. Further, at least one of NGAL, SDMA, and CysB in combination with a second biomarker of kidney function and/or injury can be applied as an early biomarker differentiating stable and progressive CKD in IRIS stage 1.

As disclosed herein, the concentration at least one of NGAL, SDMA, and CysB in combination with a second biomarker of kidney function and/or injury can be higher in animal subjects with rapid disease progression when compared with stable disease. This implies that the degree of active ongoing injury, regardless of the cause and underlying etiology, might be used as a surrogate marker for intra-renal active injury resulting in loss kidney function, which is likely irreversible (i.e., fibrosis). It is yet to be determined if therapeutic intervention might be able slow down this progression, however the fact that that a real time marker of kidney injury is available will facilitate real time assessment of various therapeutic interventions in animals with CKD.

To date, therapeutic interventions are typically monitored using functional markers, which are very slow to change. Thus a very long follow up time is required to assess these interventions during which other factors, some of which are possible to identify and control while others are not, might have also influenced the progression rate. Due to the very long period of time required to assess the effect of therapeutic interventions in dogs with early kidney disease, such studies are difficult and cost prohibitive to conduct. Yet these interventions are likely more relevant at the early stages of the disease before most of the kidney has undergone substantial irreversible changes. In this context, real time markers such as NGAL, SDMA, and CysB in combination with a second biomarker of kidney function and/or injury are valuable and will allow better understanding of the intra-renal processes governing CKD and the potential interventions to ameliorate these processes.

In some further aspects and embodiments, the disclosure provides for methods, assays, kits and multiplex point-of-care diagnostics that comprise measuring the level of at least one of NGAL, SDMA, and CysB in combination with a second biomarker of kidney function and/or injury in a biological sample from a subject. Thus, as aspects and embodiments in accordance with the disclosure comprise measuring at least two biomarkers associated with kidney function and/or injury, wherein at least one of the at least two biomarkers comprises NGAL, or at least one of the two biomarkers comprises SDMA, or at least one of the two biomarkers comprises CysB. In some further embodiments, the at least two biomarkers comprise NGAL and one or more of SDMA, cystatin B (CysB), cystatin A (CysA), fibroblast growth factor 23 (FGF-23), creatinine, clusterin, albumin, microalbumin, one or more electrolytes selected from sodium (Na+), potassium (K+) calcium (Ca2+), or magnesium (Mg2+), blood urea nitrogen (BUN), phosphorus, or inosine. In some further embodiments, the at least two biomarkers comprise SDMA and one or more of NGAL, cystatin B (CysB), cystatin A (CysA), fibroblast growth factor 23 (FGF-23), creatinine, clusterin, albumin, microalbumin, one or more electrolytes selected from sodium (Na+), potassium (K+) calcium (Ca2+), or magnesium (Mg2+), blood urea nitrogen (BUN), phosphorus, or inosine. In some further embodiments, the at least two biomarkers comprise CysB and one or more of SDMA, NGAL, cystatin A (CysA), fibroblast growth factor 23 (FGF-23), creatinine, clusterin, albumin, microalbumin, one or more electrolytes selected from sodium (Na+), potassium (K+) calcium (Ca2+), or magnesium (Mg2+), blood urea nitrogen (BUN), phosphorus, or inosine.

In some embodiments the disclosure provides for methods, assays, kits and multiplex point-of-care diagnostics that can determine kidney function and injury status in an animal subject, comprising measuring the level of at least one of NGAL, SDMA, and CysB and one or more of NGAL, SDMA, CysB, cystatin A (CysA), fibroblast growth factor 23 (FGF-23), creatinine, clusterin, albumin, microalbumin, one or more electrolytes selected from sodium (Na+), potassium (K+) calcium (Ca2+), or magnesium (Mg2+), blood urea nitrogen (BUN), phosphorus, or inosine.

In some embodiments the disclosure provides for methods, assays, kits and multiplex point-of-care diagnostics that can differentiate stable versus progressive CKD (e.g., in Stage I CKD) in an animal subject, comprising measuring the level of at least one of NGAL, SDMA, and CysB and one or more of NGAL, SDMA, CysB, cystatin A (CysA), fibroblast growth factor 23 (FGF-23), creatinine, clusterin, albumin, microalbumin, one or more electrolytes selected from sodium (Na+), potassium (K+) calcium (Ca2+), or magnesium (Mg2+), blood urea nitrogen (BUN), phosphorus, or inosine.

In some embodiments the disclosure provides for methods, assays, kits and multiplex point-of-care diagnostics that can determine stable CKD in an animal subject, comprising measuring the level of at least one of NGAL, SDMA, and CysB and one or more of NGAL, SDMA, CysB, cystatin B (CysB), cystatin A (CysA), fibroblast growth factor 23 (FGF-23), creatinine, clusterin, albumin, microalbumin, one or more electrolytes selected from sodium (Na+), potassium (K+) calcium (Ca2+), or magnesium (Mg2+), blood urea nitrogen (BUN), phosphorus, or inosine.

In some embodiments the disclosure provides for methods, assays, kits and multiplex point-of-care diagnostics that can determine progressive CKD in an animal subject, comprising measuring the level of at least one of NGAL, SDMA, and CysB and one or more of NGAL, SDMA, CysB, cystatin B (CysB), cystatin A (CysA), fibroblast growth factor 23 (FGF-23), creatinine, clusterin, albumin, microalbumin, one or more electrolytes selected from sodium (Na+), potassium (K+) calcium (Ca2+), or magnesium (Mg2+), blood urea nitrogen (BUN), phosphorus, or inosine.

In some embodiments the disclosure provides for methods, assays, kits and multiplex point-of-care diagnostics that provide for early detection of CKD in an animal subject, comprising measuring the level of at least one of NGAL, SDMA, and CysB and one or more of NGAL, SDMA, CysB, cystatin B (CysB), cystatin A (CysA), fibroblast growth factor 23 (FGF-23), creatinine, clusterin, albumin, microalbumin, one or more electrolytes selected from sodium (Na+), potassium (K+) calcium (Ca2+), or magnesium (Mg2+), blood urea nitrogen (BUN), phosphorus, or inosine.

In some embodiments the disclosure provides for methods, assays, kits and multiplex point-of-care diagnostics that provide for early staging of CKD in an animal subject, comprising measuring the level of at least one of NGAL, SDMA, and CysB and one or more of NGAL, SDMA, CysB, cystatin B (CysB), cystatin A (CysA), fibroblast growth factor 23 (FGF-23), creatinine, clusterin, albumin, microalbumin, one or more electrolytes selected from sodium (Na+), potassium (K+) calcium (Ca2+), or magnesium (Mg2+), blood urea nitrogen (BUN), phosphorus, or inosine. In yet further embodiments, the at least two biomarkers comprise NGAL and CysB.

In yet further embodiments, the at least two biomarkers comprise NGAL and SDMA.

In yet further embodiments, the at least two biomarkers comprise NGAL and fibroblast growth factor 23 (FGF-23).

In yet further embodiments, the at least two biomarkers comprise NGAL and creatinine.

In yet further embodiments, the at least two biomarkers comprise NGAL and clusterin.

In yet further embodiments, the at least two biomarkers comprise NGAL and albumin.

In yet further embodiments, the at least two biomarkers comprise NGAL and microalbumin.

In yet further embodiments, the at least two biomarkers comprise NGAL and cystatin A (CysA).

In yet further embodiments, the at least two biomarkers comprise NGAL and one or more electrolytes selected from sodium (Na+), potassium (K+) calcium (Ca2+), or magnesium (Mg2+).

In yet further embodiments, the at least two biomarkers comprise NGAL and blood urea nitrogen (BUN).

In yet further embodiments, the at least two biomarkers comprise NGAL and phosphorus.

In yet further embodiments, the at least two biomarkers comprise NGAL and inosine.

In yet further embodiments, the at least two biomarkers comprise CysB and NGAL.

In yet further embodiments, the at least two biomarkers comprise CysB and SDMA.

In yet further embodiments, the at least two biomarkers comprise CysB and fibroblast growth factor 23 (FGF-23).

In yet further embodiments, the at least two biomarkers comprise CysB and creatinine. In yet further embodiments, the at least two biomarkers comprise CysB and clusterin.

In yet further embodiments, the at least two biomarkers comprise CysB and albumin.

In yet further embodiments, the at least two biomarkers comprise CysB and microalbumin.

In yet further embodiments, the at least two biomarkers comprise CysB and cystatin A (CysA).

In yet further embodiments, the at least two biomarkers comprise CysB and one or more electrolytes selected from sodium (Na+), potassium (K+) calcium (Ca2+), or magnesium (Mg2+).

In yet further embodiments, the at least two biomarkers comprise CysB and blood urea nitrogen (BUN).

In yet further embodiments, the at least two biomarkers comprise CysB and phosphorus.

In yet further embodiments, the at least two biomarkers comprise CysB and inosine.

In yet further embodiments, the at least two biomarkers comprise SDMA and CysB.

In yet further embodiments, the at least two biomarkers comprise SDMA and fibroblast growth factor 23 (FGF-23).

In yet further embodiments, the at least two biomarkers comprise SDMA and creatinine.

In yet further embodiments, the at least two biomarkers comprise SDMA and clusterin.

In yet further embodiments, the at least two biomarkers comprise SDMA and albumin.

In yet further embodiments, the at least two biomarkers comprise SDMA and microalbumin.

In yet further embodiments, the at least two biomarkers comprise SDMA and cystatin A (CysA).

In yet further embodiments, the at least two biomarkers comprise SDMA and one or more electrolytes selected from sodium (Na+), potassium (K+) calcium (Ca2+), or magnesium (Mg2+).

In yet further embodiments, the at least two biomarkers comprise SDMA and blood urea nitrogen (BUN).

In yet further embodiments, the at least two biomarkers comprise SDMA and phosphorus.

In yet further embodiments, the at least two biomarkers comprise SDMA and inosine.

In some of the above aspects and embodiments, one or both of creatinine and/or SDMA in combination with NGAL can be used to assess the disease progression rate (e.g., slow, moderate, or high progression rate). Percent change or absolute change in creatinine or SDMA concentration can be used to define the progression, however the time during which these changes occur is critical to determine the progression rate (i.e., an increase in creatinine of 0.5 mg/dL might define a dog as rapidly progressive if occurring over 2 months and slowly progressive if occurring over 3 years). For example, in some example embodiments, a slope of at least 3 time points over at least 3 months can analyze any time effect and assess the progression rate.

In some aspects and embodiments that comprise NGAL in combination with uCysB and/or clusterin, the uCysB and/or clusterin can be used for the detection of intrarenal injury in animals with CKD and a potential surrogate marker for the progression rate.

In some of the above aspects and embodiments the methods, assays, kits and multiplex point-of-care diagnostics, provide for early prophylactic and/or therapeutic interventions that can slow, delay, or arrest progression of CKD and improve patient outcomes and quality of life.

### Stable Chronic Kidney Disease and Progressive Kidney Disease

The assays, diagnostic platforms, kits, compositions and methods described herein can be used to identify progressive and stable CKD and/or differentiate progressive CKD from stable CKD in animal subjects (e.g., mammals such as canines, felines, and humans) that have been diagnosed with CKD or that are suspected of having CKD. The advancement of progressive CKD and stable CKD can also be monitored. Progressive CKD and stable CKD can result in (1) decreased kidney function as compared to healthy subjects; or (2) physical damage to the kidneys; or (3) both. In some embodiments of these aspects, CKD (both progressive and stable) does not include cancer (e.g., renal or bladder cancer). Though referred to as “stable CKD,” stable CKD can progress through IRIS CKD stages I-IV. However, stable CKD that progresses through the four stages can be contrasted with progressive CKD, where symptoms can progress or worsen at a faster rate over time than symptoms of stable CKD.

Surprisingly, it has been determined that the use of at least one of NGAL, SDMA, and CysB in combination with a second biomarker of kidney function and/or injury can determine, without the use of two or more tests over time, if a CKD subject has stable or progressive CKD.

In some embodiments, a subject with stable CKD will have no substantial change in functional kidney markers such as NGAL, SDMA, CysB, clusterin, creatinine, or SDMA over time (e.g., over 1, 2, 3, 4, 5, 6, 7, 8, 9, 10, 11, 12, 18, 24, 36, months or more (and any range between about 1 and about 36 months, e.g., about 1 to about 3, about 2 to about 4, about 3 to about 6, about 6 to about 12, or about 1-36 months). No substantial change in functional kidney markers means less than about 2, 5, 10, 20, or 25 percent increase in the amount of functional kidney markers in a CKD patient. An increase in the amount of functional kidney markers in progressive CKD can be an increase of about 21, 25, 30, 35, 40% or more over time (e.g., over 5 days, 1, week, 2 weeks, 3 weeks, 1, 2, 3, 4, 5, 6, 7, 8, 9, 10, 11, 12, 18, 24, 36 months or more (and any range between about 5 days and about 36 months, e.g., about 1 to about 3, about 2 to about 4, about 3 to about 6, about 6 to about 12, or about 1-36 months).

In embodiments the disclosure provides methods of determining status of kidney function in an animal subject diagnosed with CKD, wherein the subject has stable CKD or progressive CKD by determining in a sample from the subject the level of at least one of NGAL, SDMA, and CysB in combination with a second biomarker of kidney function and/or injury. In further embodiments the method can be performed at a single time point. In some further embodiments, the method can be performed at multiple time points (e.g., two or more time points separated by days, weeks, or months). In some embodiments, the subject is diagnosed with stable CKD wherein the subject has elevated levels of NGAL, relative to normal. In some embodiments, the CKD subject is in IRIS Stage I or has an SDMA value of up to 20. In some embodiments the CKD subject has a urine Cystatin B level of above about 50, 60, 70, 80, 90, 100 ng/ml or more of Cystatin B (or any range between about 50 and 100 ng/ml or more (e.g., about 50-100 ng/ml or more, about 50-80 ng/ml or more, about 70-100 ng/ml or more)), and is diagnosed with progressive CKD. In such embodiments, such levels can be considered “elevated amounts” of Cystatin B. In those embodiments wherein the CKD subject has a urine level of above about 250, 260, 270, 280, 290, 300, 310, 320, 330, 340, 350 ng/ml or more of clusterin (or any range between about 250 and 350 ng/ml or more (e.g., about 250-300 ng/ml or more, about 275-325 ng/ml or more, about 325-350 ng/ml or more)), the subject is diagnosed with progressive CKD, and in those embodiments, such levels are considered “elevated amounts” of clusterin.

In some embodiments the disclosure provides methods of determining if a subject diagnosed with CKD has stable CKD by determining levels of at least one of NGAL, SDMA, and CysB in combination with a second biomarker of kidney function and/or injury. In further embodiments the method can be performed at a single time point. In some further embodiments, the method can be performed at multiple time points (e.g., two or more time points separated by days, weeks, or months). In some embodiments, the subject is diagnosed with stable CKD wherein the subject has an elevated level of NGAL relative to normal. In yet further embodiments, the mammalian CKD subject is in stable CKD or IRIS Stage I and comprises an SDMA value of up to 20. In yet further embodiments, the mammalian CKD subject is in progressive CKD and comprises a urine Cystatin B level of about 100 ng/ml or more of Cystatin B. In yet further embodiments, the mammalian CKD subject is diagnosed with stable CKD wherein the subject comprises a urine Cystatin B level of about 100, 90, 80, 70, 60, 50, 40, 30, 20 ng/ml or less (or any range between about 100 and 20 ng/mL).

Prior methods comprising of SDMA, clusterin, Cystatin B, and/or creatinine at a single time point does not provide the same results as the use of at least one of NGAL, SDMA, and CysB in combination with a second biomarker of kidney function and/or injury as described herein.

### Polypeptides

Prior research and discovery work by IDEXX Laboratories, Inc. related to various markers of kidney function and injury including, for example, SDMA, cystatin B, and clusterin has been described in U.S. Pat. Publ. 2023/0243852 (Methods and compositions for differentiating progressive chronic kidney disease from stable chronic kidney disease); U.S. Pat. No. 10,725,052 (Methods and compositions for the detection and diagnosis of renal disease and periodontal disease); U.S. Pat. No. 10,436,797 (Markers for renal disease); and U.S. Pat. Publ. 2018/0142009 (Specific Detection of Clusterin isoforms).

### Neutrophil Gelatinase-Associated Lipocalin (NGAL).

The neutrophil gelatinase-associated lipocalin gene (Ngal, also known as lcn2) is known to be upregulated following ischemic and nephrotoxic kidney injuries in humans. Further studies have confirmed that NGAL is expressed in the kidney early and robustly following ischaemic or nephrotoxic AKI in other animal models. The NGAL protein is readily detectable in biological samples (e.g., blood and urine) shortly after onset of kidney injury and has become a reliable biomarker of AKI (e.g., severity of AKI, early indicator of AKI), in animal subjects. Further, urine NGAL when analyzed in combination with creatinine (urine NGAL-to-creatinine ratio (UNCR)), has been shown to be useful in differentiating AKI from CKD.

In accordance with various embodiments of the disclosure NGAL can comprise a human NGAL polypeptide, a canine NGAL polypeptide, and/or a feline NGAL polypeptide sequence, or fragments thereof. In some embodiments of the disclosure NGAL can comprise a polynucleotide sequence that encodes a human NGAL polypeptide, a canine NGAL polypeptide, and/or a feline NGAL polypeptide sequence, or fragments thereof. Non-limiting examples of NGAL polypeptides include:

Human NGAL
(SEQ ID NO. 1)
MVPLGLLWLG LALLGALHAQ AQDSTSDLIP APPLSKVPLQ
QNFQDNQFQG KWYVVGLAGN AILREDKDPQ KMYATIYELK
EDKSYNVTSV LERKKKCDYW IRTFVPGCQP GEFTLGNIKS
YPGLTSYLVR VVSTNYNQHA MVFFKKVSQN REYFKITLYG
RTKELTSELK ENFIRESKSL GLPENHIVFP VPIDQCIDG
Canine NGAL
(SEQ ID NO. 2)
MTQVLLWLGL ALLGSLQVQT QDSTPSLIPA PPPLKVPLOP
DEQHDQFQGK WYVIGIAGNI LKKEGHGQLK MYTTTYELKD
DQSYNVTSTL LRNERCDYWN RDFVPSFQPG QFSLGDIQLY
PGVQSYLVQV VATNYNQYAL VYFRKVYKSQ EYFKITLYGR
TKELPLELKK EFIRFAKSIG LTEDHIIFPV PIDQCIDE
Feline NGAL
(SEQ ID NO. 3)
MALGILWLGL ALLGALQTHA QDSTPNLIPA PPLLLVPVEP
DFQNEQFQGK WYFLGLAGNG FNKEKHRRMK MYIANYELNE
DNSYNVTSTV AWNQTCHPST KIFLPNLHLG QFNLGNIERY
TGIQNYTSKV VTTDYNQFAI LYFKKVHDNQ EYIKVILYGR
TKEVPSVPKA IFISFIKSLG LTDDHIIFPI PNDECMDK

The methods, assays, kits and multiplex point-of-care diagnostics in accordance with the disclosure can comprise human NGAL polypeptide, canine NGAL polypeptide, and/or feline NGAL polypeptide sequence, or fragments thereof.

### Symmetric Dimethylarginine (SDMA)

SDMA is the structural isomer of the endogenous nitric oxide synthetase (NOS) inhibitor asymmetric dimethylarginine (ADMA). Both ADMA and SDMA derive from intranuclear methylation of L-arginine residuals and are released into the cytoplasm after proteolysis. SDMA is produced by protein-arginine methyltransferase 5 (PRMT 5) and PRMT 7. Proteins carrying methylarginines, such as SDMA, monomethylarginine and ADMA, play a role in RNA processing, protein shuttling and signal transduction (Bedford and Richard, Mol. Cell, 2005 Apr. 29, 18(3): 263-72). Free SDMA resulting from the degradation of such methylated proteins is mainly eliminated by renal excretion, whereas ADMA is largely metabolized. ADMA is strongly correlated with risk factors for coronary artery disease (CAD) such as hypertension, hypercholesterolemia, hyperhomocysteinemia, insulin resistance, age, and mean arterial pressure. SDMA is correlated with parameters of renal function, such as glomerular filtration rate (GFR), inulin clearance, and creatinine clearance.

### Cystatin B (CysB)

Cystatin B (CysB) is an intracellular protein belonging to the family of cysteine protease inhibitors. CysB is an intracellular protein and thus is not circulating at high serum concentrations. Cystatins A & B are members of family 1 of the cystatin superfamily and are relatively small proteins with around 11 kDa in size. In humans, these proteins are monomeric and about 11 kDa in size. They are not glycosylated and do not have the disulfide bridges seen in other cystatin superfamilies. They also lack signal sequences and are generally intra-cellular proteins confined to the cell. See, Ochieng & Chaudhuri, J Health Care Poor Underserved 2010, 21(1 Suppl): 51. Some amount of Cystatin B is present in extracellular fluids including human urine. Cystatin B has been shown to inhibit members of the lysosomal cysteine proteinases, cathepsin family, specifically cathepsin B, H, and L. See Green et al., Biochem J 1984 218:939; D'Amico et al., J Transl Med 2014, 12:350; Jarvinen & Rinne, Biochim Biophys Acta 1982, 708:210-217. Sequences of canine and feline cystatins, and fragments thereof are known. Non-limiting examples are disclosed in U.S. Publication SU 2023/024852 (e.g., UniProtKB-P25473; NCBI XP_023094432.1), and which is incorporated herein by reference.

### Clusterin or Apolipoprotein J

Clusterin (Apolipoprotein J) is a 75-80 kDa disulfide linked heterodimeric protein. Clusterin is part of many physiological processes including sperm maturation, lipid transportation, complement inhibition, tissue remodeling, membrane recycling, stabilization of stressed proteins, and promotion of inhibition of apoptosis. Clusterin polypeptides can be detected using any suitable method, including for example immunoassays. In some embodiments a combination of lectins and anti-clusterin antibodies and lectins can be used to detect clusterin. See e.g., US Pat. Publ. 20180142009. Lectins can be used that specifically bind to carbohydrates on human, canine, feline, equine, bovine, ovine, or simian clusterin isoforms. Lectins can also be used that specifically bind one or more plasma, serum, or kidney clusterin isoforms and that do not bind other clusterin isoforms.

Lectins are proteins that recognize and bind specific monosaccharide or oligosaccharide structures (carbohydrates). A lectin usually contains two or more binding sites for carbohydrate units. The carbohydrate-binding specificity of a certain lectin is determined by the amino acid residues that bind the carbohydrate. The binding strength of lectins to carbohydrates can increase with the number of molecular interactions. The dissociation constant for binding of lectins to carbohydrates is about K_{d} of 10⁻⁵ to 10⁻⁷.

Lectins can be labeled with any type of label known in the art, including, for example, fluorescent, chemiluminescent, radioactive, enzyme, colloidal metal, radioisotope and bioluminescent labels.

In some embodiments, lectins can be used that specifically bind kidney specific clusterin and that do not specifically bind plasma or serum clusterin. In some embodiments, lectins that specifically bind N-acetylglucosamine are useful in the methods. Such lectins include, for example, WGA (wheat germ agglutinin), WGA1, WGA2, WGA3, sWGA, DSL lectin (Datura stramonium lectin), mannose binding lectin, PHA-L (Phaseolus vulgaris leucoagglutanin), PHA-E (Phaseolus vulgaris erythoagglutanin), and LEL (Lycopersicon esculentum (Tomato) lectin). Other lectins that can be used include, for example jacalin, STL lectin (Solanum tuberosum), LCA lectin (Lens culinaris), PSA lectin (Pisum sativum agglutinin), ECL lectin (Erythina cristagalli), RCA lectin (Ricin communis), DBA lectin (Dolichos biflorus), SBA lectin (soybean), and CONA lectin (concanavlin). Lectins are commercially available from, e.g., Vector Laboratories.

In some embodiments clusterin can be detected with a combination of an anti-clusterin antibody or specific binding fragment thereof and a one or more lectins that specifically bind clusterin, e.g., a kidney specific clusterin isoform. See, e.g., US Pat. Publ. 20180142009. Some non-limiting examples of anti-clusterin antibodies are Clusterin Canine, Sheep Polyclonal Antibody from BioVendor Laboratory Medicine, Inc. Commercial feline antibodies for clusterin can be, for example, but not limited to Clusterin/APOJ Mouse anti-Human, Bovine, Canine, Feline, Porcine, DyLight™ 650, Clone: Hs-3, Novus Biologicals™. In some embodiments, clusterin can be detected by contacting a sample with one or more antibodies or antigen binding fragments thereof that specifically bind clusterin and one or more lectins that specifically bind to carbohydrate moieties of kidney specific clusterin and that do not specifically bind to carbohydrate moieties of other clusterin isoforms (e.g., plasma clusterin, serum clusterin, or bloodborne, non-kidney specific clusterin). Complexes of kidney specific clusterin, the one or more antibodies or antigen binding fragments thereof that specifically bind clusterin, and the one or more lectins can be detected. The lectins can specifically bind N-acetylglucosamine. Lectins can be, for example, Phaseolus vulgaris leucoagglutanin (PHA-L), wheat germ agglutinin (WGA), WGA1, WGA2, WGA3, sWGA, Phaseolus vulgaris agglutinin-E (PHA-E), Lycopersicon esculentum lectin (LEL), Datura stramonium lectin (DSL), Pisum sativum agglutinin (PSA), or Dolichos biflorus lectin (DBA), for example. The one or more antibodies or antigen binding fragments thereof can be immobilized to a support. The sample and detectably labeled one or more lectins can be added to the support. The lectins can be immobilized to a support. The sample and detectably labeled one or more antibodies or antigen binding fragments thereof can be added to the support. The one or more antibodies or antigen binding fragments thereof, the one or more lectins (e.g., plasma clusterin, serum clusterin, or bloodborne, non-kidney specific clusterin), or both can be labeled with a detectable label.

The canine (UniProtKB-P25473) and feline (UniProtKB-M3WKP2) clusterin precursor polypeptides are known in the art (see e.g., US PGPUB 2023/0243852, incorporated herein by reference in its entirety).

Methods are provided herein for the detection and quantification of at least one of NGAL, SDMA, or CysB polypeptides and fragments thereof, in combination with at least one additional biomarker of kidney function and/or injury. A polypeptide fragment of a biomarker can consist of less than about 95, 90, 80, 70, 60, 50, 40, 35, 30, 25, 20, 15, 10 (or any range between about 10 and about 95) contiguous amino acids, depending on the particular biomarker. In one embodiment a polypeptide fragment consists of more than about 10, 15, 20, 25, 30, 35, 40, 50, 60, 70, 80, 90, or 95 contiguous amino acids. In one embodiment, a polypeptide or fragment thereof is non-naturally occurring.

In accordance with the above embodiments, a “polypeptide” is a polymer of three or more amino acids covalently linked by amide bonds. A polypeptide can be post-translationally modified. A purified polypeptide is a polypeptide preparation that is substantially free of cellular material, other types of polypeptides, chemical precursors, chemicals used in synthesis of the polypeptide, or combinations thereof. A polypeptide preparation that is substantially free of cellular material, culture medium, chemical precursors, chemicals used in synthesis of the polypeptide has less than about 30%, 20%, 10%, 5%, 1% or more of other polypeptides, culture medium, chemical precursors, and/or other chemicals used in synthesis. Therefore, a purified polypeptide is about 70%, 80%, 90%, 95%, 99% or more pure.

Thus, the term “polypeptides” can refer to one or more of one type of polypeptide (a set of polypeptides). “Polypeptides” can also refer to mixtures of two or more different types of polypeptides (i.e., a mixture of polypeptides that includes but is not limited to full-length protein, truncated polypeptides, or polypeptide fragments). The terms “polypeptides” or “polypeptide” can each also mean “one or more polypeptides.”

A polypeptide variant or differs by about, for example, 1, 2, 3, 4, 5, 6, 7, 8, 9, 10, or more amino acid residues (e.g., amino acid additions, substitutions or deletions) from a naturally occurring polypeptide or a fragment thereof. Where this comparison requires alignment, the sequences are aligned for maximum homology. The site of variation can occur anywhere in the polypeptide.

Variant polypeptides can generally be identified by modifying one of the polypeptide sequences described herein, and evaluating the properties of the modified polypeptide to determine if it is a biological equivalent. A variant is a biological equivalent if it reacts substantially the same as a polypeptide described herein in an assay such as an immunohistochemical assay, an enzyme-linked immunosorbent Assay (ELISA), a turbidimetric immunoassay, a particle-enhanced turbidimetric immunoassay, a radioimmunoassay (RIA), immunoenzyme assay or a western blot assay, e.g., has 90-110% of the activity of the original polypeptide. In one embodiment, the assay is a competition assay wherein the biologically equivalent polypeptide is capable of reducing binding of the polypeptide described herein to a corresponding reactive antigen or antibody by about 80, 95, 99, or 100%. An antibody that specifically binds a corresponding polypeptide also specifically binds the variant polypeptide.

Variant polypeptides are at least about 80%, or about 81, 82, 83, 84, 85, 86, 87, 88, 89, 90, 91, 92, 93, 94, 95, 96, 97, 98, or 99% identical to the reference polypeptide sequence. For example, a variant polypeptide of SEQ ID NOs: 1-32 can be about at least 99.5%, 99%, 98%, 97%, 96%, 95%, 94%, 90%, 87%, 84%, or 81% identical to the reference sequence. Variant polypeptides have one or more conservative amino acid variations or other minor modifications and retain biological activity, i.e., are biologically functional equivalents to the reference sequence. A biologically active equivalent has substantially equivalent function when compared to the corresponding polypeptide.

Methods of introducing a mutation into an amino acid sequence are well known to those skilled in the art. See, e.g., Ausubel (ed.), Current Protocols in Molecular Biology, John Wiley and Sons, Inc. (1994); Maniatis et al., Molecular Cloning: A Laboratory Manual, Cold Spring Harbor laboratory, Cold Spring Harbor, N.Y. (1989)). Mutations can also be introduced using commercially available kits such as “QuikChange™ Site-Directed Mutagenesis Kit” (Stratagene). The generation of a functionally active variant polypeptide by replacing an amino acid that does not influence the function of a polypeptide can be accomplished by one skilled in the art.

The variant polypeptides can have conservative amino acid substitutions at one or more predicted non-essential amino acid residues. A conservative substitution is one in which an amino acid is substituted for another amino acid that has similar properties, such that one skilled in the art of peptide chemistry would expect the secondary structure and hydropathic nature of the polypeptide to be substantially unchanged. In general, the following groups of amino acids represent conservative changes: (1) ala, pro, gly, glu, asp, gln, asn, ser, thr; (2) cys, ser, tyr, thr; (3) val, ile, leu, met, ala, phe; (4) lys, arg, his; and (5) phe, tyr, trp, his. In one embodiment a polypeptide has about 1, 2, 3, 4, 5, 10, 20 or less conservative amino acid substitutions.

The terms “sequence identity” or “percent identity” are used interchangeably herein. To determine the percent identity of two polypeptide molecules or two polynucleotide sequences, the sequences are aligned for optimal comparison purposes (e.g., gaps can be introduced in the sequence of a first polypeptide or polynucleotide for optimal alignment with a second polypeptide or polynucleotide sequence). The amino acids or nucleotides at corresponding amino acid or nucleotide positions are then compared. When a position in the first sequence is occupied by the same amino acid or nucleotide as the corresponding position in the second sequence, then the molecules are identical at that position. The percent identity between the two sequences is a function of the number of identical positions shared by the sequences (i.e., % identity=number of identical positions/total number of positions (i.e., overlapping positions)×100). In some embodiments the length of a reference sequence aligned for comparison purposes is at least 80% of the length of the comparison sequence, and in some embodiments is at least 90% or 100%. In an embodiment, the two sequences are the same length.

Ranges of desired degrees of sequence identity are approximately 80% to 100% and integer values in between. Percent identities between a disclosed sequence and a claimed sequence can be at least 80%, at least 83%, at least 85%, at least 90%, at least 95%, at least 98%, at least 99%, at least 99.5%, or at least 99.9%. In general, an exact match indicates 100% identity over the length of a reference sequence.

Polypeptides and polynucleotides that are sufficiently similar to polypeptides and polynucleotides described herein (e.g., NGAL, Cystatin B, SDMA, or clusterin polypeptides) can be used herein. Polypeptides and polynucleotides that are about 90, 91, 92, 93, 94 95, 96, 97, 98, 99 99.5% or more identical to polypeptides and polynucleotides described herein can also be used herein.

For example, a polynucleotide can have 80% 85%, 90%, 91%, 92%, 93%, 94%, 95%, 96%, 97%, 98%, 99% or more identity at least one of NGAL, SDMA, or CysB and at least one other biomarker of kidney function.

A polypeptide or antibody can be covalently or non-covalently linked to an amino acid sequence to which the polypeptide or antibody is not normally associated with in nature. Additionally, a polypeptide or antibody can be covalently or non-covalently linked to compounds or molecules other than amino acids. For example, a polypeptide or antibody can be linked to an indicator reagent, an amino acid spacer, an amino acid linker, a signal sequence, a stop transfer sequence, a transmembrane domain, a protein purification ligand, or a combination thereof. In one embodiment a protein purification ligand can be one or more C amino acid residues at, for example, the amino terminus or carboxy terminus of a polypeptide. An amino acid spacer is a sequence of amino acids that are not usually associated with a polypeptide or antibody in nature. An amino acid spacer can comprise about 1, 5, 10, 20, 100, or 1,000 amino acids.

A polypeptide can be produced recombinantly. A polynucleotide encoding a polypeptide can be introduced into a recombinant expression vector, which can be expressed in a suitable expression host cell system using techniques well known in the art. A variety of bacterial, yeast, plant, mammalian, and insect expression systems are available in the art and any such expression system can be used. Optionally, a polynucleotide encoding a polypeptide can be translated in a cell-free translation system. Polypeptides can be lyophilized, desiccated, or dried, for example freeze-dried.

### Polynucleotides

In various embodiments of the above aspects, the disclosure relates to one or more isolated polynucleotide that encodes the one or more of the polypeptides disclosed herein. Polynucleotides contain less than an entire genome and can be single- or double-stranded nucleic acids. A polynucleotide can be RNA, DNA, cDNA, genomic DNA, chemically synthesized RNA or DNA or combinations thereof. The polynucleotides can be purified free of other components, such as proteins, lipids and other polynucleotides. For example, the polynucleotide can be 50%, 75%, 90%, 95%, 96%, 97%, 98%, 99%, or 100% purified. In one embodiment the polynucleotides encode a polypeptide or fragments thereof.

Polynucleotides can comprise other nucleotide sequences, such as sequences coding for linkers, signal sequences, TMR stop transfer sequences, transmembrane domains, or ligands useful in protein purification such as glutathione-S-transferase, histidine tag, and Staphylococcal protein A.

Polynucleotides can be isolated. An isolated polynucleotide is a naturally-occurring polynucleotide that is not immediately contiguous with one or both of the 5′ and 3′ flanking genomic sequences that it is naturally associated with. An isolated polynucleotide can be, for example, a recombinant DNA molecule of any length, provided that the nucleic acid sequences naturally found immediately flanking the recombinant DNA molecule in a naturally-occurring genome is removed or absent. Isolated polynucleotides also include non-naturally occurring nucleic acid molecules.

Degenerate nucleotide sequences encoding polypeptides are also contemplated herein. Degenerate nucleotide sequences are polynucleotides that encode a polypeptide as described herein or fragments thereof, but differ in nucleic acid sequence from the wild-type polynucleotide sequence, due to the degeneracy of the genetic code. Complementary DNA (cDNA) molecules, species homologs, and variants of polynucleotides that encode biologically functional polypeptides also contemplated herein.

Polynucleotides can comprise coding sequences for naturally occurring polypeptides or can encode altered sequences that do not occur in nature. If desired, polynucleotides can be cloned into an expression vector comprising expression control elements, including for example, origins of replication, promoters, enhancers, or other regulatory elements that drive expression of the polynucleotides in host cells. An expression vector can be, for example, a plasmid, such as pBR322, pUC, or ColE1, or an adenovirus vector, such as an adenovirus Type 2 vector or Type 5 vector. Optionally, other vectors can be used, including but not limited to Sindbis virus, simian virus 40, alphavirus vectors, poxvirus vectors, and cytomegalovirus and retroviral vectors, such as murine sarcoma virus, mouse mammary tumor virus, Moloney murine leukemia virus, and Rous sarcoma virus. Minichromosomes such as MC and MC1, bacteriophages, phagemids, yeast artificial chromosomes, bacterial artificial chromosomes, virus particles, virus-like particles, cosmids (plasm ids into which phage lambda cos sites have been inserted) and replicons (genetic elements that are capable of replication under their own control in a cell) can also be used.

Methods for preparing polynucleotides operably linked to an expression control sequence and expressing them in a host cell are well-known in the art. See, e.g., U.S. Pat. No. 4,366,246. A polynucleotide is operably linked when it is positioned adjacent to or close to one or more expression control elements, which direct transcription and/or translation of the polynucleotide.

Polynucleotides can be used, for example, as probes or primers, for example, PCR primers, to detect the presence of polynucleotides in a test sample, such as a biological sample. Probes are molecules capable of interacting with a target nucleic acid, typically in a sequence specific manner, for example, through hybridization. Primers are a subset of probes that can support an enzymatic manipulation and that can hybridize with a target nucleic acid such that the enzymatic manipulation occurs. A primer can be made from any combination of nucleotides or nucleotide derivatives or analogs available in the art that do not interfere with the enzymatic manipulation.

A probe or primer can be about 6, 7, 8, 9, 10, 11, 12, 13, 14, 15, 16, 17, 18, 19, 20, 25, 30, 40, 50, 60, 70, 80, 90, 100, 125, 150, 175, 200 or more contiguous nucleotides that encode polypeptides described herein.

### Antibodies

Antibodies and specific binding fragments thereof include antibody molecules that specifically bind to NGAL polypeptides. In some embodiments the disclosure further provides for one or more additional antibody molecules that specifically bind to at least one additional polypeptide that is a biomarker of kidney function or injury status. In non-limiting embodiments, the one or more additional antibody can specifically bind to Cystatin B, SDMA, FGF-23, creatinine, clusterin, albumin, microalbumin, or another protein, or fragments thereof, associated with either kidney function and/or kidney injury. An antibody can specifically bind multiple polypeptides. The term “antibodies” also includes any type of antibody molecule or specific binding fragment or molecule that specifically binds one or more NGAL polypeptides or fragments thereof, or one or more or Cystatin B, SDMA, FGF-23, creatinine, clusterin, albumin, microalbumin, or another protein, or fragments thereof, associated with either kidney function and/or kidney injury. An antibody can be naturally occurring, non-naturally occurring, synthetic, or genetically engineered. The terms “antigen-binding portion” of an antibody, “antigen-binding fragment” of an antibody, and the like, as used herein, include any naturally occurring, enzymatically obtainable, synthetic, or genetically engineered polypeptide, glycoprotein or immunoglobulin that specifically binds at least one of NGAL, SDMA, or CysB polypeptides to form a complex.

An antibody or specific binding fragment thereof binds to an epitope of a polypeptide described herein. An antibody can be made in vivo in suitable laboratory animals or in vitro using recombinant DNA techniques. Means for preparing and characterizing antibodies are well known in the art. See, e.g., Dean, Methods Mol. Biol. 80:23-37 (1998); Dean, Methods Mol. Biol. 32:361-79 (1994); Baileg, Methods Mol. Biol. 32:381-88 (1994); Gullick, Methods Mol. Biol. 32:389-99 (1994); Drenckhahn et al. Methods Cell. Biol. 37:7-56 (1993); Morrison, Ann. Rev. Immunol. 10:239-65 (1992); Wright et al. Crit. Rev. Immunol. 12:125-68 (1992). For example, polyclonal antibodies can be produced by administering a polypeptide described herein to an animal, such as a human or other primate, mouse, rat, rabbit, guinea pig, goat, pig, dog, cow, sheep, donkey, or horse. Serum from the immunized animal is collected and the antibodies are purified from the plasma by, for example, precipitation with ammonium sulfate, followed by chromatography, such as affinity chromatography. Techniques for producing and processing polyclonal antibodies are known in the art.

An antibody can be any isotype including IgG (IgG1, IgG2, IgG2a, Ig2b, IgG3, IgG4), IgM, IgA (IgA1 and IgA2), IgD, and IgE.

An antibody can be a monoclonal antibody, a polyclonal antibody, a chimeric antibody, or specific binding fragments thereof. A monoclonal antibody is an antibody obtained from a group of substantially homogeneous antibodies. A group of substantially homogeneous antibodies can contain a small amount of mutants or variants. Monoclonal antibodies are highly specific and interact with a single antigenic site. Each monoclonal antibody typically targets a single epitope, while polyclonal antibody populations typically contain various antibodies that target a group of diverse epitopes. Monoclonal antibodies can be produced by many methods including, for example, hybridoma methods (Kohler and Milstein, Nature 256:495, 1975), recombination methods (U.S. Pat. No. 4,816,567), and isolation from phage antibody libraries (Clackson et al., Nature 352:624-628, 1991; Marks et al., J. Mol. Biol. 222:581-597, 1991).

Chimeric antibodies or antigen-binding portions thereof have a part of a heavy chain and/or light chain that is derived from a specific species or a specific antibody class or subclass, and the remaining portion of the chain is derived from another species, or another antibody class or subclass. See e.g., Morrison, Science 229:1202 (1985); Oi et al., BioTechniques 4:214 (1986); Gillies et al., J. Immunol. Methods 125:191-202 (1989); U.S. Pat. Nos. 5,807,715; 4,816,567; and 4,816,397.

Chimeric antibodies can be produced using a variety of techniques including, for example, CDR-grafting (EP 239,400; PCT publication WO 91/09967; U.S. Pat. Nos. 5,225,539; 5,530,101; and 5,585,089), veneering or resurfacing (EP 592, 106; EP 519,596; Padlan, Molecular Immunology 28:489-498 (1991); Studnicka et al., Protein Engineering 7(6): 805-814 (1994); Roguska et al., PNAS 96:969-973 (1994)), and chain shuffling (U.S. Pat. No. 5,565,332).

In one embodiment, a chimeric antibody can comprise variable and constant regions of species that are different from each other, for example, an antibody can comprise the heavy chain and light chain variable regions of one mammal, and the heavy chain and light chain constant regions from a different animal (such as mouse, rabbit, canine, feline, or human). The chimeric antibody can comprise additional amino acid acids that are not included in the CDRs introduced into the recipient antibody, nor in the framework sequences. These amino acids can be introduced to more accurately optimize the antibody's ability to recognize and bind to an antigen. For example, as necessary, amino acids in the framework region of an antibody variable region can be substituted such that the CDR of a reshaped antibody forms an appropriate antigen-binding site. See Sato et al., Cancer Res. (1993) 53:851-856.

Non-limiting examples of specific binding portions or fragments of antibodies include: Fab fragments; Fab′ fragments, Fab′-SH fragments, F(ab′)₂ fragments; Fd fragments; Fv fragments; single-chain Fv (scFv) molecules; sdAb fragments (nanobodies); Fab-like antibodies (an antigen-binding fragment containing variable regions of a heavy chain and light chain that is equivalent to Fab fragments that are obtained by papain digestion); F(ab′)₂-like antibodies (an antigen-binding fragment containing two antigen-binding domains that is equivalent to F(ab′)₂ fragments that are obtained by pepsin digestion), multispecific antibodies prepared from antibody fragments, diabody, bispecific antibody, multifunctional antibody, humanized antibody, caninized antibody, human antibody, canine antibody, feline antibody, murine antibody, rabbit antibody, synthetic antibody, CDR-grafted antibody, and minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, CDR-grafted antibodies, diabodies, triabodies, tetrabodies, minibodies, nanobodies (e.g., monovalent nanobodies, bivalent nanobodies), single-chain (Fv)₂ (sc(Fv)₂); divalent (sc(Fv)₂); tetravalent ([sc(Fv)₂]₂) scFV antibodies, and small modular immunopharmaceuticals (SMIPs), and shark variable IgNAR domains, are also considered “antigen-binding fragments or portions,” as used herein.

“Specifically binds,” “specifically bind,” or “specific for” means that a first antigen, e.g., a targeted polypeptide, recognizes and binds to an antibody described herein with greater affinity than to other, non-specific molecules. “Specifically binds,” “specifically bind” or “specific for” also means a first antibody, e.g., an antibody raised against a target polypeptide (e.g., NGAL, SDMA, CysB, etc.), recognizes and binds to the sequence with greater affinity than to other non-specific molecules. A non-specific molecule is an antigen that shares no common epitope with the first antigen. Specific binding can be tested using, for example, an enzyme-linked immunosorbent assay (ELISA), a radioimmunoassay (RIA), a turbidimetric immunoassay, a particle-enhanced turbidimetric immunoassay, or a western blot assay, or other suitable assay.

### NGAL Abs

In accordance with the various aspects and embodiments of the disclosure any commercially available antibody that specifically binds to at least a first biomarker NGAL, SDMA, or CysB polypeptide or fragment thereof can be used in the methods, assays, kits, compositions, and point-of-care diagnostics described herein. Similarly antibodies that specifically bind NGAL, SDMA, or CysB can be generated by methods known in the art and/or as generally described herein.

Antibodies that specifically bind mammalian (e.g., human, canine, and feline) NGAL, SDMA, or CysB are known in the art. In some embodiments, commercial antibodies that bind to canine or feline NGAL can be used. Similarly, commercial antibodies for other markers of kidney function or kidney injury are also known in the art. Antibodies in accordance with the disclosure specifically bind at least one first biomarker (e.g., NGAL, SDMA, and/or CysB) polypeptides and fragments thereof that are present in a sample, such as a serum, blood, plasma, cells, tissue, saliva, plaque, crevicular fluid, gingival biopsy, tongue swab, or urine sample from an animal. In some particular embodiments, the sample can comprise serum, blood, plasma, or urine. In some embodiments, the disclosure provides reagents and methods for identifying renal disease, as discussed herein, in a mammal, and more particularly, in dogs, cats and humans. In certain embodiments, the disclosure provides methods for providing a diagnosis and prognosis for a renal subject or patient. As disclosed herein, identifying one or more relevant marker polypeptides described herein in patient samples can be an independent predictor of disease (likelihood of developing disease, presence of disease, progression of disease, etc.) or as an identifier of disease stage (e.g., stages 1-5). This disclosure advantageously permits diagnosis and identification of kidney disease stage at early stage (e.g., prior to stage three) and is not limited by patient age or body mass. Accordingly, additional embodiments of the disclosure are directed to using said renal patient prognosis determined using the biomarkers (e.g., polypeptides) described herein to select appropriate renal therapies.

As referred to herein, the term “immunological reagent(s)” is intended to encompass antisera and antibodies, particularly monoclonal antibodies, as well as fragments thereof (including F(ab), F(ab)₂, F(ab)′ and Fᵥ fragments). Also included in the definition of immunological reagent are chimeric antibodies, humanized antibodies, and recombinantly-produced antibodies and fragments thereof. Immunological methods used in conjunction with the reagents of the disclosure include direct and indirect (for example, sandwich-type) labeling techniques, immunoaffinity columns, immunomagnetic beads, fluorescence activated cell sorting (FACS), enzyme-linked immunosorbent assays (ELISA), radioimmune assay (RIA), as well as peroxidase labeled secondary antibodies that detect the primary antibody.

The immunological reagents of the disclosure are preferably detectably-labeled, most preferably using fluorescent labels that have excitation and emission wavelengths adapted for detection using commercially-available instruments such as and most preferably fluorescence activated cell sorters. Examples of fluorescent labels useful in the practice of the invention include phycoerythrin (PE), fluorescein isothiocyanate (FITC), rhodamine (RH), Texas Red (TX), Cy3, Hoechst 33258, and 4′,6-diamidino-2-phenylindole (DAPI). Such labels can be conjugated to immunological reagents, such as antibodies and most preferably monoclonal antibodies using standard techniques (see, e.g., Maino et al., 1995, Cytometry 20:127-133).

An immunoassay can utilize one antibody or several antibodies. An immunoassay can use, for example, a monoclonal antibody specific for one epitope, a combination of monoclonal antibodies specific for epitopes of one polypeptide, monoclonal antibodies specific for epitopes of different polypeptides, polyclonal antibodies specific for the same antigen, polyclonal antibodies specific for different antigens, or a combination of monoclonal and polyclonal antibodies. Immunoassay protocols can be based upon, for example, competition, direct reaction, or sandwich type assays using, for example, labeled antibody. Antibodies can be labeled with any type of label known in the art, including, for example, fluorescent, chemiluminescent, radioactive, enzyme, colloidal metal, radioisotope and bioluminescent labels.

Antibodies or antigen-binding fragments thereof can be bound to a support and used to detect the presence or amount of polypeptides present in samples in certain diseases and conditions. Supports include, for example, glass, polystyrene, polypropylene, polyethylene, dextran, nylon, amylases, natural and modified celluloses, polyacrylamides, agaroses and magletite. Antibodies or antigen-binding fragments thereof can be lyophilized, desiccated, or dried, for example, freeze-dried.

Antibodies can further be used to isolate polypeptides by immunoaffinity columns. The antibodies can be affixed to a solid support by, for example, absorption or by covalent linkage so that the antibodies retain their immunoselective activity. Optionally, spacer groups can be included so that the antigen binding site of the antibody remains accessible. The immobilized antibodies can then be used to specifically bind the target polypeptide(s) or fragments thereof from a biological sample, including but not limited to saliva, plaque, crevicular fluid, gingival biopsy, tongue swab, serum, blood, and urine.

Antibodies can also be used in immunolocalization studies to analyze the presence and distribution of a polypeptide described herein during various cellular events or physiological conditions. Antibodies can also be used to identify molecules involved in passive immunization and to identify molecules involved in the biosynthesis of non-protein antigens. Identification of such molecules can be useful in vaccine development. Antibodies, including, for example, monoclonal antibodies and single chain antibodies, can be used to monitor the course of amelioration of certain diseases or conditions. By measuring the increase or decrease in the amount of target polypeptide(s), or fragments thereof in a test sample from an animal, it can be determined whether a particular therapeutic regiment aimed at ameliorating the disorder is effective. Antibodies can be detected and/or quantified using for example, direct binding assays such as RIA, ELISA, or western blot assays.

### Methods of Detection and Quantification of Polypeptides

Detection of antibody-antigen complexes may be achieved through a variety of techniques well known in the art, such as, for example, turbidimetry, enzymatic labeling, radiolabeling, luminescence, or fluorescence. Immunoassay methodologies are known by those of ordinary skill in the art and are appreciated to include, but not limited to, radioimmunoassay (RIA), enzyme immunoassays (EIA), fluorescence polarization immunoassays (FPIA), microparticle enzyme immunoassays (MEIA), enzyme multiplied immunoassay technology (EMIT) assays, immuno turbidometric or agglutination assays, colloidal gold based immunoassays including lateral flow devices and chemiluminescent magnetic immunoassays (CMIA). In RIA, an antibody or antigen is labeled with radioactivity and used in a competitive or noncompetitive format. In EIA, an antibody or antigen is labeled with an enzyme that converts a substrate to a product with a resulting signal that is measured, such as a change in color. In FPIA, an antigen is labeled with fluorescent label and competes with unlabeled antigen from the specimen. The amount of analyte measured is inversely proportional to the amount of signal measured. In MEIA, a solid phase microparticle is coated with antibodies against an antigen of interest and is used to capture the analyte. The antibody for detection is labeled with an enzyme as in the EIA method. The concentration of analyte measured is proportional to the amount of signal measured. In CMIA, a chemiluminescent label is conjugated to the antibody or antigen, and produces light when combined with its substrate. CMIA can be configured in a competitive or noncompetitive format, and yields results that are inversely or directly proportional to the amount of analyte present, respectively.

As described herein, antibodies or specific binding fragments thereof can be detectably-labeled with, for example, fluorescent labels that have excitation and emission wavelengths adapted for detection using commercially-available instruments such as fluorescence activated cell sorters. Examples of fluorescent labels include phycoerythrin (PE), fluorescein isothiocyanate (FITC), rhodamine (RH), Texas Red (TX), Cy3, Hoechst 33258, and 4′,6-diamidino-2-phenylindole (DAPI). Such labels can be conjugated to antibodies using standard techniques (Maino et al., 1995, Cytometry 20:127-133).

In some embodiments, the disclosure provides methods for detecting and quantifying NGAL and one or more other biomarker polypeptides in a sample, for example, a mammalian urine sample. Methods for detecting and quantifying polypeptides include, for example, quantitative nuclear magnetic resonance (qNMR), amino acid analysis (AAA), chromatographic (HPLC) mass balance assay, liquid chromatography-mass spectrometry (LC-MS), and mass spectrometry. qNMR utilizes its unique ability to achieve equal magnitude of response from magnetic nuclei, such as ¹H, independent of chemical structure. AAA is based on quantifying stable amino acids following peptide hydrolysis. HPLC selectively quantifies an analyte of interest against a reference standard of the same analyte. In LC-MS, liquid chromatography (LC) separates the sample components and then introduces them to the mass spectrometer (MS). The MS creates and detects charged ions. The LC/MS data may be used to provide information about the molecular weight, structure, identity, and quantity of specific sample components. A mass spectrum obtained by mass spectrometry is a plot of the ion signal as a function of the mass-to-charge ratio. These spectra are used to determine the elemental or isotopic signature of a sample, the masses of polypeptides, and to elucidate the chemical identity or structure of polypeptides. Immunoassays (IAs) and competitive IAs can also be used to quantify polypeptides.

As discussed above, polypeptides can also be detected and quantified using, e.g., antibodies or specific binding fragments thereof (e.g., polyclonal antibodies, monoclonal antibodies, and specific binding fragments thereof or a combination) in immunoassays. Such methods can comprise contacting a sample with one or more antibodies or specific binding fragments thereof specific for a target sequence (e.g., NGAL, SDMA, or CysB polypeptide) under conditions suitable for formation of complexes of the target polypeptides and the one or more antibodies or specific binding fragments thereof. The complexes of polypeptides and the one or more antibodies or specific binding fragments are detected and quantified.

The use of reagent-impregnated test strips in specific binding assays is also known. In such procedures, a test sample is applied to one portion of the test strip and is allowed to migrate or wick through the strip material. Thus, the analyte to be detected or measured passes through or along the material, possibly with the aid of an eluting solvent which can be the test sample itself or a separately added solution. The analyte migrates into a capture or detection zone on the test strip, wherein a complementary binding member to the analyte is immobilized. The extent to which the analyte becomes bound in the detection zone can be determined with the aid of the conjugate which can also be incorporated in the test strip or which can be applied separately. In one embodiment, an antibody specific for NGAL, SDMA, or CysB is immobilized on a solid support at a distinct location. Following addition of the sample, detection of NGAL, SDMA, or CysB-antibody complexes on the solid support can be by any means known in the art. For example, U.S. Pat. No. 5,726,010, which is incorporated herein by reference in its entirety, describes an example of a lateral flow device, the SNAP® immunoassay device (IDEXX Laboratories).

As used herein, a microparticle refers generally to a particle that can be detected and analyzed (e.g., by flow cytometry or other analytical techniques). The term “microparticles” includes nanoparticles, nanobeads, microspheres, microbeads, nanowafers, microwafers, and other particles that are detectable. In embodiments, the microparticles may include a substrate or core and a surface functional group coupled to the substrate. In embodiments, the substrate may be another microparticle such as, for example, a microbead.

In some embodiments, the microparticles are labeled with one or more colored or fluorescent dyes. Microparticles useful in accordance with the disclosure (including microparticles labeled with colored or fluorescent dyes) are known in the art and are commercially available. Microparticles in accordance with the disclosure, including those labeled with colored or fluorescent dyes, can be prepared by methods including, but not limited to, those described in U.S. Pat. Nos. 4,267,234, 4,552,812, 5,194,300, 5,073,498, 5,981,180, 6,599,331, 7,745,091, 8,148,139, and 8,614,852 (among others), which are incorporated herein by reference in their entirety.

In embodiments, subsets of microparticles in a population of microparticles may be distinguished from other subsets (if any) based on more than one detectable parameter. In embodiments, the detectable parameter comprises fluorescence signal (e.g., intensity, wavelength maxima/minima), size, affinity, and/or shape of the particle.

Microparticles comprise any material as generally known in the art. Examples of such materials include, but are not limited to: styrene, brominated polystyrene, polyacrylic acid, polyacrylonitrile, polyacrylamide, polyacrolein, polybutadiene, polydimethylsiloxane, polyisoprene, polyurethane, polyvinyl acetate, polyvinyl chloride, polyvinyl pyridine, polyvinylbenzyl chloride, polyvinyltoluene, polyvinylidene chloride, polydivinylbenzene, polymethyl methacrylate, latex, carbohydrates (e.g., carboxymethyl cellulose, hydroxyethyl cellulose), agar, gelatin, protein polymers, polypeptides, eukaryotic and prokaryotic cells, lipids, metals, resins, latex, rubber, silicones (e.g., polydimethyl diphenylsiloxane), glass, ceramics, charcoal, kaolinite, bentonite, or combinations thereof. In embodiments, microparticles may have other surface functional groups to facilitate their attachment, adsorption and/or labeling. These groups may include, for example, carboxylic esters, alcohols, ureas, aldehydes, amines, sulfur oxides, nitrogen oxides, or halides. In some embodiments, the particles may include superparamagnetic particles, magnetic particles, or magnetizable particles.

In some embodiments, the microparticles comprise a paramagnetic, bar-coded bead containing one or more identifying features (such as a unique bar code, a color, a shape, an alphanumeric symbol, and/or the like), and/or other particles such as, for example, microbeads, microparticles, micropellets, microwafers, microparticles containing one or more identifying features (such as a bar code, a color, a shape, an alphanumeric symbol, and/or the like), paramagnetic microparticles, paramagnetic microparticles containing one or more bar codes, and/or beads containing one or more nickel bar codes. In such embodiments, the particles may be magnetic or paramagnetic.

Thus, in some embodiments of the disclosure, detection technologies employ magnetic particles or micro beads, for example, superparamagnetic iron oxide impregnated polymer beads. These beads are associated with, for example, a specific binding partner for the analyte. The beads bind with the target analytes in the sample being tested and are then typically isolated or separated out of solution magnetically. Once isolation has occurred, other testing may be conducted, including observing particular images or labels, whether directly optically or by means of a camera.

In various embodiments, assay methods used in conjunction with the antibodies described herein can include direct and indirect labeling techniques, immunoaffinity columns, immunomagnetic beads, fluorescence activated cell sorting (FACS), enzyme-linked immunosorbent assays (ELISA), radioimmune assay (RIA), agglutination assays nephelometric assays, quantitative nephelometric assays, western blot, IFA, hemagglutination (HA), turbidimetric immunoassay, particle-enhanced turbidimetric immunoassay, fluorescence polarization immunoassay (FPIA), and microtiter plate assays (any assay done in one or more wells of a microtiter plate). One assay comprises a reversible flow chromatographic binding assay, for example a SNAP® assay. See e.g., U.S. Pat. No. 5,726,010.

In some embodiments, a point-of-care (PoC) diagnostic kit can be used for the measurement of plasma biomarkers. In further embodiments, the PoC kit comprises the Triage® NGAL Device (Biosite Incorporated), which can be performed on the Triage Meter. This PoC kit requires microliter quantities of whole blood or plasma and can be deployable directly to the point of patient care.

In some embodiments, a urine immunoassay can be used in a standardized clinical platform setting. In further embodiments, the standardized clinical platform comprises the ARCHITECT® analyzer (Abbott Diagnostics). This analyzer provides non-biased measurements of urine NGAL and requires microliters of sample (e.g., 150 microlitres of urine).

In accordance with the various aspects described herein, any known, developed, and/or commercial multiplex assay system, kit, or device can be employed and adapted for use with the methods and compositions described herein, including, for example analyzers comprising the SNAP system, the Element system (e.g., Element i+, Element DCX, Element DC5X), the Vetscan/rotor system, the SMT-120VP system, the DRI-CHEM NX system, the Architect system, or the Indiko system.

In some embodiments, assays, including multiplex assays, can comprise solid phases or substrates or can be performed by immunoprecipitation or any other methods that do not utilize solid phases. Where a solid phase or substrate is used, one or more polypeptides or antibodies (or specific binding fragments thereof) are directly or indirectly attached to a solid support or a substrate such as a microtiter well, magnetic bead, non-magnetic bead, column, matrix, membrane, fibrous mat composed of synthetic or natural fibers (e.g., glass or cellulose-based materials or thermoplastic polymers, such as, polyethylene, polypropylene, or polyester), sintered structure composed of particulate materials (e.g., glass or various thermoplastic polymers), or cast membrane film composed of nitrocellulose, nylon, polysulfone or the like (generally synthetic in nature). In one embodiment a substrate is sintered, fine particles of polyethylene, commonly known as porous polyethylene, for example, 10-15 micron porous polyethylene from Chromex Corporation (Albuquerque, N. Mex.). All of these substrate materials can be used in suitable shapes, such as films, sheets, or plates, or they may be coated onto or bonded or laminated to appropriate inert carriers, such as paper, glass, plastic films, or fabrics. Suitable methods for immobilizing antibodies on solid phases include ionic, hydrophobic, covalent interactions and the like.

In one embodiment methods comprise contacting a test sample with one or a plurality of antibodies that specifically bind to two or more biomarkers of kidney function and/or kidney injury wherein the two or more biomarkers comprise NGAL, SDMA, or CysB, or fragments thereof, and another biomarker, wherein the contacting is performed under conditions that allow polypeptide/antibody complexes, i.e., immunocomplexes, to form. That is, antibodies specifically bind to one or a plurality of polypeptides of NGAL, SDMA, or CysB and at least one other biomarker or fragments thereof in the sample. One of skill in the art is familiar with assays and conditions that are used to detect antibody/polypeptide complex binding. The formation of a complex between polypeptides and antibodies in the sample is detected. The formation of antibody/polypeptide complexes is an indication that polypeptides are present in the patient sample at a certain amount.

In embodiments, a polypeptide/antibody complex is detected when an indicator reagent, such as an enzyme conjugate, which is bound to the antibody, catalyzes a detectable reaction. Optionally, an indicator reagent comprising a signal generating compound can be applied to the polypeptide/antibody complex under conditions that allow formation of a polypeptide/antibody/indicator complex. The polypeptide/antibody/indicator complex is detected. Optionally, the polypeptide or antibody can be labeled with an indicator reagent prior to the formation of a polypeptide/antibody complex. The methods can optionally comprise a positive or negative control. A positive control can contain one or more polypeptides, which will specifically bind to antibodies specific for two or more biomarkers comprising NGAL, SDMA, or CysB and another biomarker and provide a positive result. A negative control does not contain any NGAL, SDMA, or CysB and other relevant biomarkers, polypeptides or any polypeptides or other components that would specifically bind or cross-react with antibodies specific for those biomarkers.

In one embodiment, one or more antibodies are covalently or non-covalently immobilized to a solid phase or substrate. A sample potentially comprising NGAL, SDMA, or CysB (and other biomarker), polypeptide is added to the substrate. One or more antibodies (or specific binding fragments) specific for NGAL, SDMA, or CysB (and others), are added to the substrate. The antibodies can be the same antibodies used on the solid phase or can be from a different source or species and can be linked to an indicator reagent, such as an enzyme conjugate. Wash steps can be performed prior to each addition. A chromophore or enzyme substrate is added and color is allowed to develop. The color reaction is stopped and the color can be quantified using, for example, a spectrophotometer.

In other embodiments, the assays and methods can comprise second anti-species antibodies that specifically bind NGAL, SDMA, or CysB (or other biomarkers), polypeptides are added. These second anti-species antibodies are from a different species than the antibodies immobilized to the solid phase. Third anti-species antibodies are added that specifically bind the second anti-species antibodies and that do not specifically bind the antibodies immobilized to the solid phase are added. The third anti-species antibodies can comprise an indicator reagent such as an enzyme conjugate. Wash steps can be performed prior to each addition. A chromophore or enzyme substrate is added and color is allowed to develop. The color reaction is stopped and the color can be quantified using, for example, a spectrophotometer.

In one embodiment, one or more capture antibodies can specifically bind to one or more epitopes of a polypeptide described herein. The capture antibody or antibodies can be used to immobilize one or a plurality of polypeptides of NGAL, SDMA, or CysB, or fragments thereof to, for example, a solid support. One or more detection antibodies can specifically bind to the same one or more epitopes or different one or more epitopes of the polypeptides. The detection antibody can be used to detect or visualize the immobilization of the polypeptide to a solid support. This embodiment is advantageous because it is more specific and more sensitive than assays using only one antibody for both capture and detection functions.

In one embodiment of assay format, one or more antibodies can be coated onto a solid phase or substrate. A test sample suspected of containing polypeptides comprising at least two biomarkers (e.g., NGAL and one or more of Cystatin B, clusterin, SDMA, creatinine, etc.), or fragments thereof is incubated with an indicator reagent comprising a signal generating compound conjugated to an antibodies or antibody fragments specific for said polypeptides for a time and under conditions sufficient to form antigen/antibody complexes of either antibodies of the solid phase to the test sample polypeptides or the indicator reagent compound conjugated to an antibody specific for the polypeptides. The binding of the indicator reagent conjugated to anti-polypeptide antibodies to the solid phase can be quantitatively measured. A measurable alteration in the signal compared to the signal generated from a control sample or control standard indicates the presence of target polypeptides (e.g., comprising NGAL and one or more of Cystatin B, clusterin, SDMA, or creatinine, etc.) or fragments thereof. This type of assay can quantitate the amount of polypeptide in a test sample.

As discussed herein, the formation of a polypeptide/antibody complex or a polypeptide/antibody/indicator complex can be detected by, for example, radiometric, colorimetric, fluorometric, size-separation, or precipitation methods. Optionally, detection of a polypeptide/antibody complex is by the addition of a secondary antibody that is coupled to an indicator reagent comprising a signal generating compound. Indicator reagents comprising signal generating compounds (labels) associated with a polypeptide/antibody complex can be detected using the methods described above and include chromogenic agents, catalysts such as enzyme conjugates fluorescent compounds such as fluorescein and rhodamine, chemiluminescent compounds such as dioxetanes, acridiniums, phenanthridiniums, ruthenium, and luminol, radioactive elements, direct visual labels, as well as cofactors, inhibitors, magnetic particles, and the like. Examples of enzyme conjugates include alkaline phosphatase, horseradish peroxidase, beta-galactosidase, and the like. The selection of a particular label is not critical, but it will be capable of producing a signal either by itself or in conjunction with one or more additional substances.

The phrase “determining the amounts” as used herein refers to measuring or identifying the levels of one or more polypeptides in a sample. This can be accomplished by methodology well known in the art for the detection of polypeptides including using antibodies including, for example enzyme-linked immunosorbent assay (ELISA), a radioimmunoassay (RIA), a turbidimetric immunoassay, a particle-enhanced turbidimetric immunoassay, or a western blot assay, or immunohistochemistry assay. Alternatively, target polypeptides (e.g., NGAL and one or more biomarkers of kidney function and/or injury) or fragments thereof, can be determined by mass spectrometry, LC-MS, quantitative nuclear magnetic resonance (qNMR), amino acid analysis (AAA), chromatographic (HPLC) mass balance assay, or similar methods known by one of skill in the art. Determining the amount of polypeptide present in a sample is accomplished by such in vitro analysis and experimental manipulation.

### Diagnostic Methods

In various aspects and embodiments, the disclosure provides methods for diagnosing stable CKD or progressive CKD in a subject. In some embodiments, a subject can have a diagnosis of CKD, and then the amount of NGAL, SDMA, or CysB biomarker and another biomarker polypeptide of kidney function and/or injury in a sample from the CKD diagnosed subject is determined. The methods can comprise determining the amount of NGAL, SDMA, or CysB in combination with one or more other biomarker (e.g., NGAL, SDMA, Cystatin B, creatinine, clusterin, etc.) in a sample from the subject, wherein the amount of the biomarker polypeptides are determined using, for example, one or more antibodies. The amount of NGAL, SDMA, or CysB and other biomarker(s) in the sample is compared to a control sample or control standard, wherein certain levels of NGAL, SDMA, or CysB and/or other biomarker(s) in the sample compared to the control sample or control standard is an indication of either stable CKD or progressive CKD in the subject.

Some embodiments provide a method for diagnosing or detecting stable CKD. The method comprises determining the amount of NGAL, SDMA, or CysB in combination with one or more other biomarker (e.g., NGAL, SDMA, Cystatin B, creatinine, clusterin, etc.) in a sample from the subject, wherein the amount of biomarker polypeptides in the sample is compared to a control sample or control standard, wherein elevated levels of NGAL, SDMA, or CysB and one or more other biomarker polypeptides in the sample compared to the control sample or control standard is an indication of stable CKD.

Some embodiments provide a method for diagnosing or detecting progressive CKD. The method comprises determining the amount of NGAL, SDMA, or CysB in combination with one or more other biomarker (e.g., NGAL, SDMA, Cystatin B, creatinine, clusterin, etc.) in a sample from the subject, wherein the amount of biomarker polypeptides in the sample is compared to a control sample or control standard, wherein elevated levels of NGAL, SDMA, or CysB and one or more other biomarker polypeptides in the sample compared to the control sample or control standard is an indication of progressive CKD.

In some embodiments, antibody-independent methods can be used to diagnose or detect stable or progressive CKD. The method can comprise determining the amount NGAL, SDMA, or CysB polypeptides in combination with one or more other biomarker polypeptides in a sample from the subject, wherein the amount of NGAL, SDMA, or CysB and one or more other biomarker polypeptides is determined using LC-MS assay or other assays described herein. The amount of the biomarker polypeptides in the sample is compared to a control sample or control standard, wherein elevated levels of biomarker polypeptides in the sample compared to the control sample or control standard is an indication of stable CKD or progressive CKD.

In embodiments, elevated levels of NGAL polypeptides can be levels that are statistically significantly increased amounts when compared to control samples or control standards.

In certain embodiments elevated levels of Cystatin B polypeptides can be above about 50, 60, 70, 80, 90, 100 ng/ml or more of Cystatin B (or any range between about 50 and 100 ng/ml or more (e.g., about 50-100 ng/mL or more, about 50-80 ng/ml or more, about 70-100 ng/ml or more)), Control levels or control standards of Cystatin B polypeptides can be about 49, 40, 30, 25, 20, 15, 10, 5, 1 or less ng/ml.

In certain embodiments elevated levels of clusterin polypeptides can be above about 250, 260, 270, 280, 290, 300, 310, 320, 330, 340, 350 ng/ml or more of clusterin (or any range between about 250 and 350 ng/ml or more (e.g., about 250-300 ng/ml or more, about 275-325 ng/ml or more, about 325-350 ng/ml or more). Control levels or control standards of clusterin polypeptides can be about 249, 240, 230, 225, 200, 150, 100, 50 or less ng/ml.

In various embodiments, elevated levels of biomarker polypeptides can be compared to control samples or control standards that are determined using normal control subjects who do not have any type of kidney disease or condition, or bacterial infection.

Accordingly, in various embodiments, the disclosure provides methods for differentiating stable chronic kidney disease (“CKD”) from progressive CKD in a subject diagnosed with CKD by using the devices, multiplex assay systems, and/or kits in accordance with the disclosure to:
(a) determine the amount of a first biomarker as described herein in a sample from the subject at least at one first time point;
(b) determine the amount of a second biomarker as described herein in a sample from the subject at least at one first time point;
(b) compare the amount of the first and second biomarkers in the sample to a standard values indicative of a healthy subject, wherein an increase in one or both of the first and second biomarkers is an indication of progressive CKD.

In embodiments, the devices, systems, and assay methods in accordance with the disclosure can comprise a computing device as part of the device and/or multiplex system. The computing device can perform various acts and/or functions, such as those described in this disclosure. The computing device can include various components, such as a processor, a data storage unit, a communication interface, and/or a user interface. These components can be connected to each other (or to another device, system, or other entity) via one or more connection mechanisms as are generally known in the art.

In embodiments, the processor can include a general-purpose processor (e.g., a microprocessor) and/or a special-purpose processor (e.g., a digital signal processor (DSP)).

In embodiments, the data storage unit can include one or more volatile, non-volatile, removable, and/or non-removable storage components, such as magnetic, optical, or flash storage, and/or can be integrated in whole or in part with the processor. Further, the data storage unit can take the form of a non-transitory computer-readable storage medium, having stored thereon program instructions (e.g., compiled or non-compiled program logic and/or machine code) that, when executed by a processor, cause the computing device to perform one or more acts and/or functions, such as those described herein. As such, the computing device can be configured to perform one or more acts and/or functions, such as those described in this disclosure. Such program instructions can define and/or be part of a discrete software application. In some instances, the computing device can execute program instructions in response to receiving an input, such as from the communication interface and/or the user interface. In embodiments, the data storage unit can also store other types of data, such as those types described in this disclosure.

In embodiments, the communication interface can allow the computing device to connect to and/or communicate with another other entity according to one or more protocols. In one example, the communication interface can be a wired interface, such as an Ethernet interface or a high-definition serial-digital-interface (HD-SDI). In another example, the communication interface can be a wireless interface, such as a cellular or WI FI interface. In accordance with various embodiments of the disclosure, a connection can be a direct connection or an indirect connection, the latter being a connection that passes through and/or traverses one or more entities, such as such as a router, switcher, or other network device. Likewise, in embodiments of this disclosure, a transmission can be a direct transmission or an indirect transmission.

In embodiments, the user interface can facilitate interaction between the computing device and a user of the computing device, if applicable. As such, the user interface can include input components such as a keyboard, a keypad, a mouse, a touch sensitive panel, a microphone, a camera, and/or a movement sensor, all of which can be used to obtain data indicative of an environment of the computing device, and/or output components such as a display device (which, for example, can be combined with a touch sensitive panel), a sound speaker, and/or a haptic feedback system. More generally, the user interface can include hardware and/or software components that facilitate interaction between the computing device and the user of the computing device.

In embodiments, the computing device can take various forms, such as a workstation terminal, a desktop computer, a laptop, a tablet, a mobile phone, or a controller.

In accordance with some embodiments, the level of one or more other target biomarker of kidney function and/or injury in a test sample is compared the level of the same target biomarker(s) in a control sample from one or more normal control subjects. Typically, the measured control level in the control sample is then compared with the level measured in the test sample. Alternatively, the level of target biomarker(s) polypeptides in the test sample is compared to a previously determined or predefined control level (a “control standard”). For example, the control standard for NGAL, Cystatin B, clusterin, SDMA, or creatinine polypeptides can be calculated from data, such as data including the levels of NGAL, Cystatin B, clusterin, SDMA, or creatinine, in control samples from a plurality of normal or healthy control subjects. The normal or healthy control subjects and the test subject under assessment can be of the same species.

Particular embodiments provide reagents and methods for identifying certain diseases or conditions in mammals, e.g., in dogs, cats, and humans. Certain embodiments provide methods for providing a diagnosis and prognosis for CKD patients. The methods advantageously permit diagnosis and identification of progressive CKD and is not influenced or confounded by CKD patient age or body mass. Accordingly, additional embodiments are directed to using a progressive CKD diagnosis determined by the methods described herein to select appropriate therapies.

Embodiments further include methods for prognosing CKD patient health, monitoring disease progression, and/or assessing/monitoring treatment efficacy by identifying levels of NGAL polypeptides in a CKD patient sample. In some aspects, the methods can be performed at a single time point. In other aspects, the methods can be performed in multiple time points (e.g., about 2, 3, 4, 5, or more time points) to, for example, evaluate disease progression or treatment efficacy. In a particular embodiment, the methods may be performed at diagnosis and then at specific time points post-treatment wherein a specific therapy should result in a reduction or amelioration of disease progression.

The compositions and methods are more particularly described below and the Examples set forth herein are intended as illustrative only, as numerous modifications and variations therein will be apparent to those skilled in the art.

The terms used in the specification generally have their ordinary meanings in the art, within the context of the compositions and methods described herein, and in the specific context where each term is used. Some terms have been more specifically defined herein to provide additional guidance to the practitioner regarding the description of the compositions and methods.

As used herein, the term “and/or” includes any and all combinations of one or more of the associated listed items. As used in the description herein and throughout the claims that follow, the meaning of “a”, “an”, and “the” includes plural reference as well as the singular reference unless the context clearly dictates otherwise. The term “about” in association with a numerical value means that the value varies up or down by 5%. For example, for a value of about 100, means 95 to 105 (or any value between 95 and 105).

All patents, patent applications, and other scientific or technical writings referred to anywhere herein are incorporated by reference herein in their entirety. The embodiments illustratively described herein suitably can be practiced in the absence of any element or elements, limitation or limitations that are specifically or not specifically disclosed herein. Thus, for example, in each instance herein any of the terms “comprising,” “consisting essentially of,” and “consisting of” can be replaced with either of the other two terms, while retaining their ordinary meanings. The terms and expressions which have been employed are used as terms of description and not of limitation, and there is no intention that in the use of such terms and expressions of excluding any equivalents of the features shown and described or portions thereof, but it is recognized that various modifications are possible within the scope of the claims. Thus, it should be understood that although the present methods and compositions have been specifically disclosed by embodiments and optional features, modifications and variations of the concepts herein disclosed can be resorted to by those skilled in the art, and that such modifications and variations are considered to be within the scope of the compositions and methods as defined by the description and the appended claims.

Any single term, single element, single phrase, group of terms, group of phrases, or group of elements described herein can each be specifically excluded from the claims.

Whenever a range is given in the specification, for example, a temperature range, a time range, a composition, or concentration range, all intermediate ranges and subranges, as well as all individual values included in the ranges given are intended to be included in the disclosure. It will be understood that any subranges or individual values in a range or subrange that are included in the description herein can be excluded from the aspects herein. It will be understood that any elements or steps that are included in the description herein can be excluded from the claimed compositions or methods.

In addition, where features or aspects of the compositions and methods are described in terms of Markush groups or other grouping of alternatives, those skilled in the art will recognize that the compositions and methods are also thereby described in terms of any individual member or subgroup of members of the Markush group or other group.

1. A device comprising:
a solid phase having bound thereto at least a first analyte sensing reagent and a second analyte sensing reagent:
A) the first analyte sensing reagent comprising a sensing reagent or an antibody specific for a first biomarker comprising:
(i) Neutrophil Gelatinase-Associated Lipocalin (NGAL), an NGAL analog, or an antibody specific for NGAL, or any combination thereof;
(ii) Symmetrical Dimethylarginine (SDMA), an SDMA analog, or an antibody specific for SDMA, or any combination thereof; or
(ii) Cystatin B (CysB), a CysB analog, or an antibody specific for CysB, or any combination thereof;
B) the second analyte sensing reagent comprising a sensing reagent or an antibody specific for a second biomarker; and
wherein the first analyte sensing reagent and the second analyte reagent are not the same.

2. The device of claim 1 wherein the device is a cartridge comprising:
a plurality of electrodes;
at least one magnet;
a first surface of the solid phase that transports a droplet, via the plurality of electrodes, on the first surface of the solid phase of the cartridge, wherein the droplet comprises at least one paramagnetic, bar-coded bead, and wherein the plurality of electrodes is configured to transport the droplet on the first surface of the solid phase the cartridge; and
a second surface of the solid phase that immobilizes the droplet, via the at least one magnet, on the second surface, wherein the at least one magnet is configured to immobilize the droplet on the second surface of the cartridge; and
wherein the first analyte sensing reagent and the second analyte sensing reagent are not the same.

3. The device of claim 2, wherein the first surface of the cartridge and the second surface of the cartridge are the same surface.

4. The device of claim 2, wherein the first surface of the cartridge and the second surface of the cartridge are different surfaces.

5. The device of claim 1, wherein the at least one second analyte sensing reagent is bound to a second solid phase.

6. The device of claim 1, wherein the at least one second biomarker comprises NGAL, SDMA, CysB, fibroblast growth factor 23 (FGF-23), creatinine, blood urea nitrogen (BUN), clusterin, albumin, microalbumin, inosine, or an electrolyte selected from Na+, K+Ca2+, Mg2+, or phosphorus; wherein
when the first biomarker comprises NGAL, the at least one second biomarker comprises SDMA, CysB, Na+, K+Ca2+, Mg2+, fibroblast growth factor 23 (FGF-23), creatinine, blood urea nitrogen (BUN), phosphorus, clusterin, albumin, microalbumin, or inosine;
when the first biomarker comprises SDMA, the at least one second biomarker comprises NGAL, CysB, Na+, K+Ca2+, Mg2+, fibroblast growth factor 23 (FGF-23), creatinine, blood urea nitrogen (BUN), phosphorus, clusterin, albumin, microalbumin, or inosine; and
when the first biomarker comprises CysB, the at least one second biomarker comprises NGAL, SDMA, Na+, K+Ca2+, Mg2+, fibroblast growth factor 23 (FGF-23), creatinine, blood urea nitrogen (BUN), phosphorus, clusterin, albumin, microalbumin, or inosine.

7. The device of claim 1, wherein the first biomarker comprises NGAL, an NGAL analog, or an antibody specific for NGAL, or any combination thereof.

8. The device of claim 1, wherein the first biomarker comprises SDMA, an SDMA analog, or an antibody specific for SDMA, or any combination thereof.

9. The device of claim 1, wherein the first biomarker comprises CysB, a CysB analog, or an antibody specific for CysB, or any combination thereof.

10. The device of claim 1, wherein the device further comprises one or more additional solid phase having bound thereto one or more additional analyte sensing reagent or one or more additional antibody specific for one or more additional biomarker.

11. The device of claim 10, wherein the device further comprises N additional solid phases having bound thereto N additional analyte sensing reagents or N additional antibodies specific for N additional biomarkers.

12. The device of claim 11, wherein N is an integer selected from 1, 2, 3, 4, 5, 6, 7, or 8.

13. The device of claim 1, wherein the device further comprises a non-transitory computer-readable medium, having stored thereon program instructions that, upon execution by a controller, cause a controller to perform a set of operations comprising detecting a signal generated by the first analyte sensing reagent and the second analyte sensing reagent.

14. A multiplex assay system or kit for determining kidney function and injury status in an animal subject, the system or kit comprising the device of claim 1.

15. The device, multiplex assay system, or kit according to claim 14, for (i) use in determining stable chronic kidney disease in an animal subject (CKD), (ii) for use determining progressive CKD for use in early detection of CKD in the animal subject, (iii) for use in early detection of CKD in an animal subject, or (iv) for use in early staging of CKD in an animal subject.

16. A method for determining kidney function and injury status in an animal subject, the method comprising the device, multiplex assay system or kit according to claim 1, and further comprising measuring the concentration of at least a first and a second biomarker in a biological sample from the subject:
the first biomarker comprising:
Neutrophil Gelatinase-Associated Lipocalin (NGAL), an NGAL analog, or an antibody specific for NGAL, or any combination thereof;
Symmetrical Dimethylarginine (SDMA), an SDMA analog, or an antibody specific for SDMA, or any combination thereof; or
Cystatin B (CysB), a CysB analog, or an antibody specific for CysB, or any combination thereof; and
the second biomarker comprising a sensing reagent or an antibody specific for the second biomarker; and
comparing the concentration of the first and second biomarkers to one or more standard values that are associated with normal kidney function and/or CKD or AKI in the animal subject, wherein the comparing indicates whether the subject has normal kidney function or kidney injury.

17. The method of claim 16 for determining stable chronic kidney disease (CKD) in an animal subject comprising:
comparing the concentration of the first and second biomarkers to one or more standard values that are associated with normal kidney function and/or stable CKD, wherein the comparing indicates whether the subject has stable CKD.

18. The method of claim 16 for determining progressive chronic kidney disease (CKD) in an animal subject comprising:
comparing the concentration of the first and second biomarkers to one or more standard values that are associated with normal kidney function and/or progressive CKD wherein the comparing indicates whether the subject has progressive CKD.

19. The method of claim 16 for early detection of chronic kidney disease (CKD) in an animal subject comprising:
comparing the concentration of the first and the second biomarker to one or more standard values that are associated with normal renal function in the animal subject wherein the comparing identifies early CKD in the animal subject.

20. The method of claim 16 for early staging of chronic kidney disease (CKD) in an animal subject comprising:
comparing the concentration of the first and the second biomarker to one or more standard values that are associated with normal renal function in the animal subject wherein the comparing identifies the stage of CKD in the subject.

21. The method of claim 16, wherein the at least one second biomarker comprises NGAL, SDMA, CysB, fibroblast growth factor 23 (FGF-23), creatinine, blood urea nitrogen (BUN), clusterin, albumin, microalbumin, inosine, or an electrolyte selected from Na+, K+Ca2+, Mg2+, or phosphorus; wherein
when the first biomarker comprises NGAL, the at least one second biomarker comprises SDMA, CysB, Na+, K+Ca2+, Mg2+, fibroblast growth factor 23 (FGF-23), creatinine, blood urea nitrogen (BUN), phosphorus, clusterin, albumin, microalbumin, or inosine;
when the first biomarker comprises SDMA, the at least one second biomarker comprises NGAL, CysB, Na+, K+Ca2+, Mg2+, fibroblast growth factor 23 (FGF-23), creatinine, blood urea nitrogen (BUN), phosphorus, clusterin, albumin, microalbumin, or inosine; and
when the first biomarker comprises CysB, the at least one second biomarker comprises NGAL, SDMA, Na+, K+Ca2+, Mg2+, fibroblast growth factor 23 (FGF-23), creatinine, blood urea nitrogen (BUN), phosphorus, clusterin, albumin, microalbumin, or inosine.
